(19) United States
(12) Patent Application Publication
(10) Pub. No.: US 2022/0348667 A1
(43) Pub. Date: Nov. 3, 2022

(54) Specific ACKR2 Modulators for Use in Therapy

(71) Applicant: Luxembourg Institute of Health (LIH), Luxembourg (LU)

(72) Inventors: Bassam Janji, Thionville (FR); Muhammad Zaeem Noman, Thionville (FR); Andy Chevigné, Saint Léger (FR); Martyna Szpakowska, Luxembourg (LU); Guy Berchem, Leudelange (LU); Markus Ollert, Mondorf-les-Bains (LU)

(73) Assignee: Luxembourg Institute of Health (LIH), Luxembourg (LU)

(21) Appl. No.: 17/620,281

(22) PCT Filed: Jul. 15, 2020
(86) PCT No.: PCT/EP2020/070036

§ 371 Date: Dec. 17, 2021

(30) Foreign Application Priority Data
Jul. 15, 2019 (EP) 19186373.7

(51) Int. Cl. C07K 16/28; A61P 35/00

(52) U.S. Cl. CPC C07K 16/2866; A61P 35/00; C07K 2317/76

(57) ABSTRACT

The application discloses a specific ACKR2 modulator for use in the treatment of a proliferative disease or disorder in a subject and/or for use in improving the response of a subject to anticancer immunotherapy. The application further discloses pharmaceutical compositions comprising such a specific ACKR2 modulator and one or more immune checkpoint modulators, preferably one or more immune checkpoint inhibitors.

### FIELD

The invention is broadly in the medical field, and provides the use of atypical chemokine receptor 2 (ACKR2) modulating molecules in therapy, both as such and in combination with other agents.

### BACKGROUND

Cancer immunotherapy and notably immune checkpoint blockades (ICB) have emerged as promising treatment approaches for several advanced highly aggressive cancers for which conventional therapies have failed. However, recent clinical observations demonstrated that relatively few patients benefit from significant clinical remissions.

Therefore, the next challenge in the field of ICB-based cancer immunotherapies is the development of innovative strategies to be combined with ICB in order to extend their use and benefit to large number of cancer patients notably non-responder patients. Several studies claimed that the durable clinical response to immune checkpoint inhibitors is likely dependent on the infiltration of cytotoxic effector immune cells, notably T-lymphocytes and natural killer (NK) cells, into the tumor bed. It is now well established that such infiltration strikingly relies on the establishment of inflamed tumor microenvironment. Indeed, according to the inflammatory status, two major subsets of advanced solid tumors can be identified: tumors with a T cell-inflamed or “hot” microenvironment, which display a signature of a pre-existing adaptive immune response, and non-T cell-inflamed or “cold” microenvironment, which lack evidence of a pre-existing adaptive immune response. Therefore, developing innovative strategies to induce such signature may ultimately lead to reprograming “cold, immune desert” tumor microenvironment to a “hot, immune receptive” one.

In view of the above, there is an urgent need to explore new ways to develop strategies, to be combined with ICB, in order to make tumors and cancer patients responsive to ICB.

### SUMMARY

Present inventors established that ACKR2 binds and scavenges the C-X-C motif chemokine 10 (CXCL10) and that a specific ACKR2 (i.e. an ACKR2-specific) modulator that blocks the scavenging of CCL5 and/or CXCL10 can increase the infiltration of NK and CD8+ T cells into the tumor microenvironment. Furthermore, present inventors established that specific ACKR2 modulators having a high affinity and selectivity for ACKR2 can be used in the prevention or treatment of proliferative diseases or disorders and to improve the response of a subject to anticancer immunotherapy, such as to one or more immune checkpoint inhibitors.

Accordingly, a first aspect of the invention provides a specific ACKR2 modulator for use in the treatment of a proliferative disease or disorder in a subject.

In particular embodiments, said treatment comprises the administration of said specific ACKR2 modulator in combination with anticancer immunotherapy, preferably in combination with one or more immune checkpoint inhibitors.

In particular embodiments, said ACKR2 modulator specifically binds to ACKR2.

In particular embodiments, said ACKR2 modulator decreases the quantity and/or expression level of ACKR2.

In particular embodiments, said ACKR2 modulator is selected from a group consisting of a chemical substance, an antibody, an antibody fragment, an antibody-like protein scaffold, a protein or polypeptide, a peptide, a peptidomimetic, an aptamer, a photoaptamer, a spiegelmer and a nucleic acid.

In particular embodiments of the first and second aspect, said ACKR2 modulator is selected from a group consisting of an anti-ACKR2 antibody, an ACKR2-directed gene-editing system, an RNAi agent directed against ACKR2, a fragment or derivative of CCL2, a fragment or derivative of CCL3, a fragment or derivative of CCL4, a fragment or derivative of CCL5, a fragment or derivative of CCL7, a fragment or derivative of CCL8, a fragment or derivative of CCL11, a fragment or derivative of CCL13, a fragment or derivative of CCL14, a fragment or derivative of CCL17, a fragment or derivative of CCL22 and a fragment or derivative of CXCL10.

In particular embodiments, said proliferative disease or disorder is a proliferative disease or disorder selected from the group consisting of skin cancer such as melanoma, colon cancer, rectal cancer, colorectal cancer, bladder cancer, neuroblastoma, squamous cell cancer, lung cancer, cancer of the peritoneum, hepatocellular cancer, gastric or stomach cancer such as gastrointestinal cancer, pancreatic cancer, glioblastoma, cervical cancer, ovarian cancer, liver cancer, hepatoma, breast cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, head cancer and neck cancer, preferably skin cancer or colorectal cancer.

In particular embodiments, said subject is diagnosed with or assumed to have a proliferative disease or disorder which is non-responsive to anticancer immunotherapy, preferably non-responsive to immune checkpoint blockade therapy.

In particular embodiments, said treatment comprises the administration of said ACKR2 modulator in combination with anticancer immunotherapy, preferably in combination with one or more immune checkpoint inhibitors.

A second aspect of the invention provides a pharmaceutical composition comprising a specific ACKR2 modulator and one or more immune checkpoint modulators, preferably one or more immune checkpoint inhibitors, and optionally a pharmaceutically acceptable carrier.

In particular embodiments of the first and second aspect, the one or more immune checkpoint modulators, preferably the one or more immune checkpoint inhibitors, are selected from the group consisting of a Programmed Death-ligand 1 (PDL-1) inhibitor, a Programmed Death 1 (PD-1) inhibitor, a Cytotoxic T-Lymphocyte-Associated protein 4 (CTLA-4) inhibitor, a cluster of differentiation 3 (CD3) inhibitor, a NKG2A inhibitor, a immunoglobulin-like receptors (KIR) inhibitor, a cluster of differentiation 4 (CD47) inhibitor, a CD24 inhibitor, a CD73 inhibitor, a tumor necrosis factor (TNF) receptor 2 (TNFR2) inhibitor, and a signal-regulatory protein alpha (SIRPα) inhibitor, preferably a PD-1 inhibitor.

In particular embodiments of the first and second aspect, the PD-1 inhibitor is selected from the group consisting of nivolumab, pembrolizumab, pidilizumab, cemiplimab, tislelizumab, sintilimab and MEDI068.

A further aspect provides an in vitro method for identifying an agent useful as a therapeutic, such as useful for the treatment of a proliferative disease or disorder in a subject, said method comprising determining whether a test agent modulates the biological activity of ACKR2.

In particular embodiments, said method comprises a step of determining whether a test agent decreases or eliminates the biological activity of ACKR2.

In particular embodiments, said method comprises
contacting the test agent with a cell capable inducing β-arrestin 1 and/or β-arrestin 2 recruitment to ACKR2 in the presence of CCL2, CCL3, CCL4, CCL5, CCL7, CCL8, CCL11, CCL13, CCL14, CCL17, CCL22 and/or CXCL10 and measuring β-arrestin 1 and/or β-arrestin 2 recruitment to ACKR2, and
determining that the agent is useful as a therapeutic when the agent reduces or eliminates β-arrestin1 and/or β-arrestin 2 recruitment to ACKR2 in the presence of CCL2, CCL3, CCL4, CCL5, CCL7, CCL8, CCL11, CCL13, CCL14, CCL17, CCL22 and/or CXCL10.

In particular embodiments, said method further comprises determining whether the test agent specifically binds to ACKR2.

### BRIEF DESCRIPTION OF DRAWINGS

FIG. 1: β-arrestin-1 recruitment to ACKR2 and CXCR3 evaluated by the Nanoluciferace complementation-based assay. (A) β-arrestin-1 recruitment to ACKR2 induced by human CXC chemokines CXCL1, CXCL2, CXCL3, CXCL4, CXCL5, CXCL6, CXCL7, CXCL8, CXCL9, CXCL10, CXCL11, CXCL12, CXCL13, CXCL14, CXCL16, CXCL17, CCL5 and CCL2 at a single concentration of 100 nM. (B) Concentration-response curves for β-arrestin-1 recruitment to ACKR2 induced by the newly identified ligand, CXCL10 and the positive and negative control chemokines, CCL5 and CXCL11, respectively. (C) Concentration-response curves for β-arrestin-1 recruitment to CXCR3 induced by its cognate ligands, CXCL10 and CXCL11, and the negative control chemokine, CXCL12. Results are expressed as fold change over vehicle and presented as mean±SEM (n≥3). (D) β-arrestin-1 recruitment to ACKR2 monitored by NanoBRET. (E) Flow cytometry analysis of cells used in the binding studies, left panel: ACKR2 surface expression in HEK.ACKR2 (grey non-filled histogram) and the parental HEK293T cell line (grey filled histogram) evaluated using the ACKR2-specific mAb (clone 196124) or the corresponding isotype control (black non-filled histogram); right panel: CXCR3 surface expression in HEK.ACKR2 evaluated using the CXCR3-specific mAb (clone 106) (dark grey non-filled histogram) and the corresponding isotype control (black histogram). Unstained cells are represented as grey filled histogram. (F) Binding of Cy5-labelled CXCL10 to HEK.ACKR2 cells (inset) Binding competition with unlabelled chemokines. Data points represent mean±SEM of three independent experiments. *p<0.05, **p<0.01, ***p<0.001, **** p<0.0001 by Bonferroni (F) post hoc test.

FIG. 2: Specific activation of CXCR3 and ACKR2 by CXCL10. (A) β-arrestin-1 recruitment to all known chemokine receptors in response to CXCL10 (100 nM). EC₅₀ values for concentration-response curves of FIG. 1 (B-E) are indicated (nM). Data points represent mean±SEM of three independent experiments. *p<0.05, **p<0.01, ***p<0.001, **** p<0.0001 by one-way ANOVA with Dunnett post hoc test.

FIG. 3: CXCL10 scavenging by ACKR2. (A) Chemokines remaining in the supernatant following 8-hour incubation with ACKR2-positive and -negative HEK293 cells were quantified by ELISA. CCL5 (C) and CXCL11 (D) were used as positive and negative control chemokines, respectively. (B): bar graphs representing the chemokines CXCL10, CCL5 and CXCL11 remaining in the cell supernatant expressed as % of input chemokines (30 nM). Results presented as mean±SEM (n≥3). (E) HEK, HEK-ACKR2 or HEK-ACKR2 cells pre-treated with CCL5 at saturating concentration (200 nM) were stimulated for 40 minutes at 37° C. with 100 nM (Cy5)-labelled CXCL10. For each condition, the percentage of cells with a given number of distinguishable vesicle-like structures (spots), as well as the geometrical mean fluorescence intensity (MFI) for the red channel were determined (inset). Data shown are representative of three independent experiments and mean±SEM, respectively. (F) Impact of chemokine N terminal processing by dipeptidyl peptidase 4-processed (DPP4/CD26) on the activation of ACKR2. Concentration-reponse curves of processed chemokines on ACKR2 monitored by NanoBRET. (G) Impact of chemokine N terminal processing by dipeptidyl peptidase 4-processed (DPP4/CD26) on the activation of ACKR2 and related receptors CXCR3 and CCR5. Comparison of the impact of N terminal processing on the ability of CXC and CC chemokines (100 nM) to activate ACKR2, CXCR3 and CCR5. *p<0.05, **p<0.01, ***p<0.001 by two-tailed paired t test.

FIG. 4: B16-F10 melanoma cells were transfected with vector pCMV-HA ACKR2 encoding HA-tagged ACKR2. (A) The expression of Ackr2 mRNA was assessed by RT-qPCR. Results are reported as fold change (FC) compared to control condition. (B) The cell surface expression of Ackr2 was assessed as a % of live cells by flow cytometry using Ackr2 antibody (anti-chemokine receptor D6 antibody (ab38567; Abcam)). Data are represented as the average of 2 independent experiments (performed in duplicate).

FIG. 5: ELISA quantification of the secreted CCL5 (A) and CXCL10 (B) protein in the supernatant of B16-F10 cells described in FIG. 1. Results are reported in pg/ml of cell supernatant. Data are reported as the average of 2 independent experiments (performed in duplicate). Results are shown as mean±SEM (error bars). Statistically significant differences (indicated by asterisks) calculated compared to control conditions using an unpaired two-tailed Student's t-test are shown (ns=not significant, **=p<0.005).

FIG. 6: B16-F10 melanoma cells were transfected with control (sh-CT) or Ackr2 (sh-Ackr2) lentivirus sh-RNA. (A) The expression of Ackr2 mRNA was assessed by RT-qPCR. Results are reported as fold change (FC) compared to control condition. (B) The cell surface expression of Ackr2 was assessed as a % of live cells by flow cytometry using Ackr2 antibody (ab38567).

FIG. 7: ELISA quantification of the secreted CCL5 (A) and CXCL10 (B) protein in the supernatant of B16-F10 cells described in FIG. 1. Results are reported in pg/ml of cell supernatant. Data are represented as the average of 2 independent experiments (performed in duplicate). Results are shown as mean±SEM (error bars). Statistically significant differences (indicated by asterisks) calculated compared to control conditions using an unpaired two-tailed Student's t-test are shown (ns=not significant, **=p<0.005).

FIG. 8: Experimental design (A), tumor growth curve (B) and tumor weight in grams (g) (C) of control (shCT) or Ackr2-targeted (shAckr2) B16-F10 melanoma tumors in immunocompetent C57BL/6 mice. Each curve represents the average of 3 independent experiments of 5 mice per group. Results are shown as mean±SEM (error bars). Statistically significant differences (indicated by asterisks) are calculated compared to control conditions using an unpaired two-tailed Student's t-test (ns=not significant, *=p<0.05, ***=p<0.0005).

FIG. 9: Experimental design (A), tumor growth curve (B) and tumor weight in grams (g) (C) of control (shCT) or Ackr2-targeted (shAckr2) B16-F10 melanoma tumors in immunodeficient NSG mice. Each curve represents 2 independent experiments of 5 or 6 mice per group. Results are shown as mean±SEM (error bars). Statistically significant differences (indicated by asterisks) are calculated compared to control conditions using an unpaired two-tailed Student's t-test (ns=not significant).

FIG. 10: Flow cytometry quantification of CD45+ leukocytes infiltrating the control (shCT) or Ackr2-targeted (sh-Ackr2) B16-F10 melanoma tumors at day 17 in immunocompetent C57BL/6 mice. The defined subpopulations were gated and quantified in live CD45+ cells. Each dot represents one tumor. The data are reported as the average of 5 mice per group. Results are shown as mean±SEM (error bars). Statistically significant differences (indicated by asterisks) are calculated compared to control conditions using an unpaired two-tailed Student's t-test (**=p<0.005).

FIG. 11: Flow cytometry quantification of CD3+, CD4+, CD4+ effector T cells (CD4+ eff), NK cells, CD8+ T cells, and regulatory T lymphocytes (Treg) infiltrating the control (shCT) or Ackr2-targeted (shAckr2) B16-F10 melanoma tumors at day 17 in immunocompetent C57BL/6 mice. The defined subpopulations were gated and quantified in live CD45+ cells. Each dot represents one tumor. The data are reported as the average of 5 mice per group. Results are shown as mean±SEM (error bars). Statistically significant differences (indicated by asterisks) are calculated compared to control conditions using an unpaired two-tailed Student's t-test (ns=not significant, *=p<0.05, **=p<0.005 and ***=p<0.0005).

FIG. 12: CD8+ to Treg cells ratio (CD8+/Treg) in control (shCT) or Ackr2-targeted (shAckr2) B16-F10 melanoma tumors at day 17 in immunocompetent C57BL/6 mice. Each dot represents one tumor. The data are reported as the average of 5 mice per group. Results are shown as mean±SEM (error bars). Statistically significant differences (indicated by asterisks) are calculated compared to control conditions using an unpaired two-tailed Student's t-test (*=p<0.05).

FIG. 13: Quantification of the percent of CD69+ activated CD4 effector T cells (CD4 eff), NK cells (NK), CD8 T cells (CD8), infiltrating the control (shCT) or Ackr2-targeted (sh-Ackr2) B16-F10 melanoma tumors at day 17 in immunocompetent C57BL/6 mice. Each dot represents one tumor. The data are reported as the average of 5 mice per group. Results are shown as mean±SEM (error bars). Statistically significant differences (indicated by asterisks) are calculated compared to control conditions using an unpaired two-tailed Student's t-test (ns=not significant, *=p<0.05, **=p<0.005).

FIG. 14: ELISA quantification of the CCL5, CXCL10 and IFNγ secreted in the microenvironment of control (shCT) or Ackr2-targeted (shAckr2) B16-F10 melanoma tumors at day 17 in immunocompetent C57BL/6 mice. Data are reported in pg/ml standardized to excised tumor weight (g) and represented as an average of 5 tumors per group (each dot represents one mouse). All results are shown as mean±SEM (error bars). Statistically significant differences (indicated by asterisks) are calculated compared to control conditions using an unpaired two-tailed Student's t-test (*=p<0.05; **=p<0.005).

FIG. 15: The inhibition of tumor growth, observed by targeting Ackr2, is dependent on the infiltration of NK and CD8+ T cells into the tumor microenvironment. Growth curves (upper panels) and weight in grams “g” (lower panels) of sh-CT (left panels) and sh-Ackr2 (right panels) B16-F10 tumors in mice treated with control isotype (Iso), NK-depleted (αNK) or CD8-depleted (αCD8) antibodies. Data described in the figure is reported as the average of 5 mice per group. Each dot represents one mouse. Results are shown as mean±SEM (error bars). Tumor weights were assessed at day 17. Statistically significant differences (indicated by asterisks) are calculated compared to control conditions using an unpaired two-tailed Student's t-test (ns=not significant, *=p<0.05, **=p<0.005 and ***=p<0.0005).

FIG. 16: Growth curves, weight in grams “g” (upper panels), and flow cytometry quantification of CD45+, NK cells, CD8+ and CD4+ T cells (lower panels) infiltrating sh-CT and sh-Ackr2 B16-F10 tumors harvested from mice injected with either control (Iso) or CCL5 neutralizing antibody (αCCL5). Results are reported as the average of 5 mice per group. All results are shown as mean±SEM (error bars). Statistically significant differences (indicated by asterisks) are calculated compared to control conditions using an unpaired two-tailed Student's t-test (ns=not significant, *=p<0.05, and ***=p<0.0005).

FIG. 17: Targeting Ackr2 improves the therapeutic benefit of anti-PD-1 immunotherapy. A: The treatment strategy of sh-CT and sh-Ackr2 B16-F10 tumor beating mice. 0.2×10⁶ B16-F10 cells were subcutaneously injected into the right flank of syngeneic host C57BL/6 mice at day 0. After the development of palpable tumors (typically at day 9), mice were intraperitoneally (i.p.) injected with 3 doses of 100 μg anti-PD1 or control isotype. B: Upper panels: Tumor growth curves of sh-CT and sh-Ackr2 B16-F10 melanoma combined with either control isotype (Iso), or anti-PD-1 (αPD-1). Middle panels: Tumor weight (in grams, g) on day 17 of sh-CT and sh-Ackr2 B16-F10 melanoma combined with either control isotype (Iso), or anti-PD-1. Results are reported as the average of 10 mice per group from 2 independent experiments conducted with 5 mice per group. Results are shown as mean±SEM (error bars). Statistically significant differences (indicated by asterisks) are calculated using an unpaired two-tailed Student's t-test (ns=not significant, *=p<0.05, **=p<0.005 and ***=p<0.0005). Lower panels: Mice survival curves (5 mice per group) were generated from sh-CT and sh-Ackr2 tumor bearing mice treated with either control isotype (Iso) or anti-PD-1 (αPD-1). Lack of survival was defined as death or tumor size >1000 mm³. Mice survival percentage was defined using Graph Pad Prism and P values were calculated using the Log-rank (Mantel-Cox) test (*−p≤0.05, **−p≤0.01).

FIG. 18: Ability of the polyclonal anti-mACKR2 (ab1656, Abcam) to compete with CCL5 binding to ACKR2. Binding competition studies were performed in HEK.mACKR2 cells. Binding of CCL5-AF647 was evaluated by flow cytometry and represented as percentage of CCL5-AF647 bound to the receptor in the absence of antibody. Goat IgG (ab37373, Abcam) was used as negative control.

FIG. 19: Ability of the polyclonal anti-mACKR2 (ab1656, Abcam) to quantify the concentration of mCXCL10 (upper panel) and mCCL5 (lower panel) in the cell supernatant using commercially available ELISA kits (R&D Systems). Inhibition of mCXCL10 and mCCL5 scavenging by anti-mACKR2 was expressed as the percentage relative to cells incubated in the absence of the antibody. Goat IgG (ab37373, Abcam) was used as negative control.

FIG. 20: Treatment of B16-F10 melanoma tumor bearing mice with anti-Ackr2 blocking antibody improves the therapeutic benefit of anti-PD-1 immunotherapy. A: The treatment strategy of B16-F10 tumor beating mice. B: Upper panels: Tumor growth curves of B16-F10 melanoma tumors treated with control isotypesisotype (Iso), anti-Ackr2, anti-PD-1 (α-PD-1) or a combination of anti-Ackr2 and anti-PD-1 αAckr2/α-PD-1. Middle panels: Tumor weight (in grams, g) on day 17. Results are reported as the average of 10 mice per group from 2 independent experiments conducted with 5 mice per group. Results are shown as mean±SEM (error bars). Statistically significant differences (indicated by asterisks) are calculated using an unpaired two-tailed Student's t-test (ns=not significant, *=p<0.05, **=p<0.005 and ***=p<0.0005). Lower panels: Mice survival curves (5 mice per group) were generated from B16-F10 melanoma tumors treated as described in upper panels. Lack of survival was defined as death or tumor size >1000 mm³. Mice survival percentage was defined using Graph Pad Prism and P values were calculated using the Log-rank (Mantel-Cox) test (*−p<0.05, **−p<0.01).

### DESCRIPTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

Atypical chemokine receptors (ACKRs) are a small family of membrane proteins comprising four members (ACKR1, 2, 3 and 4). In contrast to classical chemokine receptors, ACKRs are unable to initiate classical signaling pathways after chemokine binding but instead modulate chemokine bioavailability by transporting them through endosomal pathway to lysosomal compartment following the recruitment of β-arrestin. ACKRs are therefore considered as scavenging receptors for chemokines and as major receptors orchestrating chemokine-driven immune and inflammatory responses. ACKR2 is a member of the ACKR family. Indeed, following chemokines binding, ACKR2 rapidly traffics from the cell surface to endosomes and chemokines can be dislodged from the receptor by the low pH in endosomes before being degraded in lysosomes. Chemokine-free ACKR2 traffics back to the cell surface to re-acquire chemokines. The repeated internalization and recycling leads to progressive depletion of extracellular chemokines. The ACKR2 internalizes through clathrin-coated pits, in a mechanism dependent on classical endosome signaling pathway involving Rab5, Vps15, Vps34, UVRAG and Beclin1 proteins. ACKR2 is able to bind inflammatory chemokines exclusively belonging to the CC subfamily including CCL2, CCL3, CCL4, CCL5, CCL7, CCL8, CCL11, CCL12, CCL13, CCL14, CCL17 and CCL22. Human ACKR2 is able to bind CCL2, CCL3, CCL4, CCL5, CCL7, CCL8, CCL11, CCL13, CCL14, CCL17 and CCL22

The present inventors have found that ACKR2 binds and scavenges the C-X-C motif chemokine 10 (CXCL10), which until now was thought to interact exclusively with C-X-C motif chemokine receptor 3 (CXCR3). CXCL10 is one of the major inflammatory chemokines involved in driving T cells into inflamed tissues and tumors. In addition, the present inventors have found that exclusively targeting ACKR2 in a subject decreases the volume and weight of tumors in a subject. More particularly, they established that a specific ACKR2 (i.e. an ACKR2-specific) modulator that blocks the scavenging of CCL5 and/or CXCL10, preferably a specific ACKR2 modulator that simultaneously blocks the scavenging of both CCL5 and CXCL10, can increase the infiltration of NK and CD8+ T cells into the tumor microenvironment. More particularly, the increased bioavailability of CCL5 and/or CXCL10 results in switching cold immune desert tumors to hot inflamed immune cell-infiltrated tumors by recruiting effector immune cells, such as CD69+NK and CD8+ T cells but also CD4+ and CD4 effector cells, to the tumor bed. Switching cold to hot tumors is of particular interest for anticancer immunotherapy, such as immune checkpoint modulation therapy, preferably immune checkpoint blockade therapy. Accordingly, specific ACKR2 modulators having a high affinity and selectivity for ACKR2 can be used in the prevention or treatment of a proliferative diseases or disorders and to improve the response of a subject to anticancer immunotherapy, such as to one or more immune checkpoint modulators, such as to one or more immune checkpoint inhibitors.

Accordingly, a first aspect provides a specific ACKR2 modulator (i.e. an ACKR2-specific), more particularly for use in therapy.

The term “specific” as used herein means that an agent binds to or influences one or more desired molecules or analytes substantially to the exclusion of other molecules which are random or unrelated, and optionally substantially to the exclusion of other molecules that are structurally related. The terms do not necessarily require that an agent binds exclusively to its intended target(s).

In particular embodiments, the specific ACKR2 modulator as described herein is not capable of binding to any other chemokine receptor than ACKR2, more particularly a chemokine receptor selected from the group consisting of C-C chemokine receptor type 1 (CCR1) (e.g. with UniProt accession number P32246), C-C chemokine receptor type 2 (CCR2) (e.g. with UniProt accession number P41597) such as CCR type 2A (CCR2A) or CCR type 2B (CCR2B), C-C chemokine receptor type 3 (CCR3) (e.g. with UniProt accession number P51677), C-C chemokine receptor type 4 (CCR4) (e.g. with UniProt accession number P51679), C-C chemokine receptor type 5 (CCR5) (e.g. with UniProt accession number P51681), C-C chemokine receptor type 6 (CCR6) (e.g. with UniProt accession number P51684), C-C chemokine receptor type 7 (CCR7) (e.g. with UniProt accession number P32248), C-C chemokine receptor type 8 (CCR8) (e.g. with UniProt accession number P51685), C-C chemokine receptor type 9 (CCR9) (e.g. with UniProt accession number P51686), C-C chemokine receptor type 10 (CCR10) (e.g. with UniProt accession number P46092), C-X-C motif chemokine receptor 1 (CXCR1) (e.g. with UniProt accession number P25024), C-X-C motif chemokine receptor 2 (CXCR2) (e.g. with UniProt accession number P25025), C-X-C motif chemokine receptor 3 (CXCR3) (e.g. with UniProt accession number P49682) such as CXCR type 3A (CXCR3A) and CXCR type 3B (CXCR3B), C-X-C motif chemokine receptor 4 (CXCR4) (e.g. with UniProt accession number P61073), C-X-C motif chemokine receptor 5 (CXCR5) (e.g. with UniProt accession number P32302), C-X-C motif chemokine receptor 6 (CXCR6) (e.g. with UniProt accession number O00574), C-X-C motif chemokine receptor 8 (CXCR8) (e.g. with UniProt accession number Q9HC97), X-C motif chemokine receptor 1 (XCR1) (e.g. with UniProt accession number P46094), C-X3-C motif chemokine receptor 1 (CX3CR1) (e.g. with UniProt accession number P49238), atypical chemokine receptor 1 (ACKR1) (e.g. with UniProt accession number Q16570), atypical chemokine receptor 3 (ACKR3) (e.g. with UniProt accession number P25106) and atypical chemokine receptor 4 (ACKR4) (e.g. with UniProt accession number Q9NPB9).

In particular embodiments, the specific ACKR2 modulator as described herein is not capable of inducing the recruitment of β-arrestin-1 and β-arrestin-2 to other chemokine receptor than ACKR2, more particularly a chemokine receptor selected from the group consisting of CCR1 (e.g. with UniProt accession number P32246), CCR2 (e.g. with UniProt accession number P41597) such as CCR type 2A (CCR2A) or CCR type 2B (CCR2B), CCR3 (e.g. with UniProt accession number P51677), CCR4 (e.g. with UniProt accession number P51679), CCR5 (e.g. with UniProt accession number P51681), CCR6 (e.g. with UniProt accession number P51684), CCR7 (e.g. with UniProt accession number P32248), CCR8 (e.g. with UniProt accession number P51685), CCR9 (e.g. with UniProt accession number P51686), CCR10 (e.g. with UniProt accession number P46092), CXCR1 (e.g. with UniProt accession number P25024), CXCR2 (e.g. with UniProt accession number P25025), CXCR3 (e.g. with UniProt accession number P49682) such as CXCR type 3A (CXCR3A) and CXCR type 3B (CXCR3B), CXCR4 (e.g. with UniProt accession number P61073), CXCR5 (e.g. with UniProt accession number P32302), CXCR6 (e.g. with UniProt accession number O00574), CXCR8 (e.g. with UniProt accession number Q9HC97), XCR1 (e.g. with UniProt accession number P46094), CX3CR1 (e.g. with UniProt accession number P49238), ACKR1 (e.g. with UniProt accession number Q16570), ACKR3 (e.g. with UniProt accession number P25106) and ACKR4 (e.g. with UniProt accession number Q9NPB9).

By means of additional guidance, atypical chemokine receptor 2 (ACKR2) is also known in the art as Chemokine-binding protein D6, chemokine receptor D6, chemokine-binding protein 2, chemokine receptor CCR-9, CMKBR9 or CCBP2. By means of an example, human ACKR2 gene is annotated under NCBI Genbank (http://www.ncbi.nlm.nih.gov/) Gene ID 1238. Human ACKR2 mRNA is annotated under NCBI Genbank accession number NM 001296.5. Nucleotides 207 (start codon) to 1361 (stop codon) of NM_001296.5 constitute the ACKR2 coding sequence. Human ACKR2 protein sequence is annotated under NCBI Genbank accession number NP_001287.2, and Uniprot (www.uniprot.org) accession number O00590.2.

Although historically (i.e. before the year 2000), ACKR2 may have been given the name CCR10 at some point after its identification as a chemokine receptor (number 10 being the first available in the CCR subfamily), ACKR2 was excluded from the CCR nomenclature of classical chemokine receptors (among others because of its inability to trigger canonical G protein-dependent signalling) and the name CCR10 has been since reattributed to another chemokine receptor that binds to chemokines CCL27 and CCL28 and signals via G proteins. Accordingly, the person skilled in the art will understand that ACKR2 (e.g. with UniProt accession number O00590.2) having ligands CCL2, CCL3, CCL4, CCL5, CCL7, CCL8, CCL11, CCL13, CCL14, CCL17 and CCL22 is different from the classical chemokine receptor CCR10 (e.g. with UniProt accession number P46092) having ligands CCL27 and CCL28.

A skilled person can appreciate that any sequences represented in sequence databases or in the present specification may be of precursors of the respective peptides, polypeptides, proteins or nucleic acids and may include parts which are processed away from mature molecules.

The term “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, that which is being modulated may be changed or altered without modulated without substantially altering other (unintended, undesired, unrelated) targets, functions, properties or processes. The term “modulator” as used herein, refers to an agent that interacts with a target either directly or indirectly. The interactions include, but are not limited to, the interactions of an agonist, partial agonist, an inverse agonist, antagonist, degrader, or combinations thereof.

By means of an example and not limitation, the ACKR2 modulator may modulate one or more aspects of the biological activity of the ACKR2 polypeptide, such as its ability to recruit β-arrestin and/or its ability to interact with endogenous or exogenous ligands.

In particular embodiments, the ACKR2 modulator as described herein inhibits, reduces and/or prevents the interaction between ACKR2 and ACKR2 endogenous or exogenous ligands, such as endogenous chemokines (e.g. CCL2, CCL3, CCL4, CCL5, CCL7, CCL8, CCL11, CCL12, CCL13, CCL14, CCL17, CCL22 and/or CXCL10, preferably CCL5 and/or CXCL10).

In particular embodiments, the ACKR2 modulator as described herein inhibits, reduces and/or prevents the interaction between human ACKR2 and human ACKR2 endogenous or exogenous ligands, such as endogenous chemokines (e.g. CCL2, CCL3, CCL4, CCL5, CCL7, CCL8, CCL11, CCL13, CCL14, CCL17, CCL22 and/or CXCL10, preferably CCL5 and/or CXCL10).

In particular embodiments, the ACKR2 modulator as taught herein inhibits, reduces and/or prevents the interaction between ACKR2 and CCL5 and/or CXCL10.

The inhibition, reduction and/or prevention of the interaction between ACKR2 and endogenous ACKR2 ligands by the ACKR2 modulator as taught herein can be determined by any means known in the art, such as competition binding assays or displacement assays.

In particular embodiments, the ACKR2 modulator as taught herein inhibits, reduces and/or prevents the interaction between ACKR2 and CCL5 and/or CXCL10 by at least 10%, at least 20%, at least 30%, at least 40%, at least 50%, at least 60%, at least 70%, at least 80%, at least 90%, at least 95%, or at least 99%, such as by 100%, preferably at least 40%.

In particular embodiments, the ACKR2 modulator is able to displace labelled CCL5 and/or CXCL10 at a concentration of less than 1 μM, more particularly less than 100 nM or less than 10 nM, such as less than 5 nM.

In particular embodiments, the ACKR2 modulator specifically binds to ACKR2.

In particular embodiments, the specific ACKR2 modulator as described herein is capable of specifically binding the conserved tyrosine motif at the N terminus of ACKR2, for example the conserved tyrosine motif at the N terminus of ACKR2 as described in Hewit et al., The N-terminal Region of the Atypical Chemokine Receptor ACKR2 Is a Key Determinant of Ligand Binding. J Biol. Chem. 2014. 289(18):12330-12342.

The terms “bind”, “interact”, “specifically bind” or “specifically interact” as used throughout this specification mean that an agent binds to or influences one or more desired molecules or analytes substantially to the exclusion of other molecules which are random or unrelated, and optionally substantially to the exclusion of other molecules that are structurally related. The terms do not necessarily require that an agent binds exclusively to its intended target(s). For example, an agent may be said to specifically bind to target(s) of interest if its affinity for such intended target(s) under the conditions of binding is at least about 2-fold greater, preferably at least about 5-fold greater, more preferably at least about 10-fold greater, yet more preferably at least about 25-fold greater, still more preferably at least about 50-fold greater, and even more preferably at least about 100-fold or more greater, such as, e.g., at least about 1000-fold or more greater, at least about 1×10⁴-fold or more greater, or at least about 1×10⁵-fold or more greater, than its affinity for a non-target molecule.

The binding or interaction between the agent and its intended target(s) may be covalent (i.e., mediated by one or more chemical bonds that involve the sharing of electron pairs between atoms) or, more typically, non-covalent (i.e., mediated by non-covalent forces, such as for example, hydrogen bridges, dipolar interactions, van der Waals interactions, and the like). Preferably, the agent may bind to or interact with its intended target(s) with affinity constant (K_{A}) of such binding K_{A}≥1×10⁶ M⁻¹, more preferably K_{A}≥1×10⁷ M⁻¹, yet more preferably K_{A}≥1×10⁸ M⁻¹, even more preferably K_{A}≥1×10⁹ M⁻¹, and still more preferably K_{A}≥1×10¹⁰ M⁻¹ or K_{A}≥1×10¹¹ M⁻¹, wherein K_{A}=[A_T]/[A][T], A denotes the agent, T denotes the intended target. Determination of K_{A} can be carried out by methods known in the art, such as for example, using equilibrium dialysis and Scatchard plot analysis.

An agent is said to “specifically bind to” a particular target when that agent has affinity for, specificity for, and/or is specifically directed against that target (i.e., against at least one part or fragment thereof).

The “specificity” of a specific ACKR2 modulator as described herein can be determined based on affinity. The “affinity” of an agent, such as a polypeptide, protein or peptide, is represented by the equilibrium constant for the dissociation of the agent and ACKR2, preferably human ACKR2 (e.g. as annotated under NCBI Genbank accession number NP_001287.2). The lower the KD value, the stronger the binding strength between the agent and ACKR2. Alternatively, the affinity can also be expressed in terms of the affinity constant (KA), which corresponds to 1/KD. A KD value greater than about 1 millimolar is generally considered to indicate non-binding or non-specific binding.

The binding of an agent, such as a polypeptide, protein or peptide, as described herein to a target and the affinity and specificity of said binding may be determined by any methods known in the art. Non-limiting examples thereof include binding competition assays using fluorescently labelled or radiolabelled ligands (e.g. fluorescently labelled or radiolabelled chemokines, such as CCL5 or CXCL10), co-immunoprecipitation, bimolecular fluorescence complementation, affinity electrophoresis, label transfer, phage display, proximity ligation assay (PLA), Tandem affinity purification (TAP), in-silico docking and calculation of the predicted Gibbs binding energy and competition binding assays.

In other particular embodiments, the specific ACKR2 modulator decreases the quantity and/or expression level of ACKR2 in a subject and/or in a sample obtained from said subject, for example in a cell, tissue or organ, preferably a tumor or tumor cell, of said subject.

The terms “quantity”, “amount” and “level” are synonymous and generally well-understood in the art. The terms as used herein may particularly refer to an absolute quantification of a molecule or an analyte, such as in a sample obtained from a subject, or to a relative quantification of a molecule or analyte, such as in a sample obtained from a subject, i.e., relative to another value such as relative to a reference value, or to a range of values indicating a base-line expression of the molecule or analyte. These reference or base-line values or ranges can be obtained from a single patient or from a group of patients.

An absolute quantity of a molecule or analyte, such as in a sample obtained from a subject, may be advantageously expressed as weight or as molar amount, or more commonly as a concentration, e.g., weight per volume or mol per volume.

A relative quantity of a molecule or analyte, such as in a sample obtained from a subject, may be advantageously expressed as an increase or decrease or as a fold-increase or fold-decrease relative to said another value, such as relative to a reference value. Performing a relative comparison between first and second parameters (e.g., first and second quantities) may but need not require first to determine the absolute values of said first and second parameters. For example, a measurement method can produce quantifiable readouts (such as, e.g., signal intensities) for said first and second parameters, wherein said readouts are a function of the value of said parameters, and wherein said readouts can be directly compared to produce a relative value for the first parameter vs. the second parameter, without the actual need first to convert the readouts to absolute values of the respective parameters.

The terms “quantity” and “expression level” of ACKR2 are used interchangeably in this specification to refer to the absolute and/or relative quantification, concentration level or amount of any such product, for example in a sample obtained from a subject.

In other particular embodiments, the specific ACKR2 modulator decreases the quantity and/or expression level of ACKR2, for example in a cell, tissue or organ, preferably a tumor or tumor cell, by at least 50%, at least 60%, at least 70%, at least 75%, at least 80%, at least 85%, at least 90%, at least 95%, at least 99%, such as 100%, preferably at least 75%, compared to a reference situation without said ACKR2 modulation.

Any existing, available or conventional separation, detection and quantification methods may be used herein to measure the presence or absence (e.g., readout being present vs. absent; or detectable amount vs. undetectable amount) and/or quantity (e.g., readout being an absolute or relative quantity, such as, for example, absolute or relative concentration) of peptides, polypeptides, proteins, or nucleic acids in cells, tissues or organs, in vitro, ex vivo or in vivo.

The level of ACKR2 at the protein level may be detected using standard quantitative protein measurement tools known in the art. Non-limiting examples include immunoassay methods, and protein immunoblotting (e.g. Western blot, dot blot).

The level of ACKR2 at the nucleic acid level, more particularly RNA level, e.g., at the level of hnRNA, pre-mRNA, mRNA, or cDNA, may be detected using standard quantitative RNA or cDNA measurement tools known in the art. Non-limiting examples include hybridisation-based analysis, microarray expression analysis, digital gene expression (DGE), RNA-in-situ hybridisation (RISH), Northern-blot analysis and the like; PCR, RT-PCR, RT-qPCR, end-point PCR, digital PCR or the like; supported oligonucleotide detection, pyrosequencing, polony cyclic sequencing by synthesis, simultaneous bi-directional sequencing, single-molecule sequencing, single molecule real time sequencing, true single molecule sequencing, hybridization-assisted nanopore sequencing, sequencing by synthesis, or the like.

In particular embodiments, the specific ACKR2 modulator acts as ACKR2 antagonist and can reduce β-arrestin-1 and/or β-arrestin-2 recruitment to ACKR2. The interaction between ACKR2 and β-arrestin-1 and/or β-arrestin-2 can be determined by any established analytical technique for determining protein-protein binding, such as co-immunoprecipitation, bimolecular fluorescence complementation, label transfer, tandem affinity purification, chemical cross-linking, fluorescence resonance energy transfer and nanoluciferase complementation assays (e.g. NanoBiT, Promega or NanoBRET, Promega), for instance using ACKR2 C-terminally fused to SmBiT and β-arrestin-1 or β-arrestin-2 N-terminally fused to LgBiT. Protein binding assays may be performed in a cell-free system or in a cell lysate or in isolated or cultured cells or in an isolated or cultured tissue.

In other particular embodiments, the specific ACKR2 modulator acts as ACKR2 agonist and can induce β-arrestin-1 and/or β-arrestin-2 recruitment to ACKR2, provided that it retains the ability to block the binding of CCL5 and/or CXCL10.

In particular embodiments, the specific ACKR2 modulator as described herein has a potency for ACKR2 that is characterized by an EC₅₀ of 10 nM or less, 9 nM or less, 8 nM or less, 7 nM or less, 6 nM or less, 5 nM or less, 4 nM or less, 3 nM or less, 2 nM or less, 1 nM or less, 0.95 nM or less, 0.90 nM or less, 0.85 nM or less, 0.80 nM or less, 0.75 nM or less, 0.70 nM or less or 0.65 nM or less, preferably an EC₅₀ of 5 nM or less, more preferably an EC₅₀ of 1 nM or less. The EC₅₀ in the context of the present invention can be determined based on β-arrestin recruitment assay. β-arrestin recruitment can be determined by any methods known in the art such as by nanoluciferase complementation assays (e.g. NanoBiT, Promega or NanoBRET, Promega), for instance using ACKR2 C-terminally fused to SmBiT and the β-arrestin N-terminally fused to LgBiT.

In particular embodiments, the ACKR2 modulator is selected from a group consisting of a chemical substance (e.g. small molecule), an antibody, an antibody fragment, an antibody-like protein scaffold, a protein or polypeptide, a peptide, a peptidomimetic, an aptamer, a photoaptamer, a spiegelmer and a nucleic acid, or a combination of any two or more thereof.

The term “protein” as used throughout this specification generally encompasses macromolecules comprising one or more polypeptide chains, i.e., polymeric chains of amino acid residues linked by peptide bonds. The term may encompass naturally, recombinantly, semi-synthetically or synthetically produced proteins. The term also encompasses proteins that carry one or more co- or post-expression-type modifications of the polypeptide chain(s), such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes protein variants or mutants which carry amino acid sequence variations vis-à-vis a corresponding native proteins, such as, e.g., amino acid deletions, additions and/or substitutions. The term contemplates both full-length proteins and protein parts or fragments, e.g., naturally-occurring protein parts that ensue from processing of such full-length proteins.

The term “polypeptide” as used throughout this specification generally encompasses polymeric chains of amino acid residues linked by peptide bonds. Hence, especially when a protein is only composed of a single polypeptide chain, the terms “protein” and “polypeptide” may be used interchangeably herein to denote such a protein. The term is not limited to any minimum length of the polypeptide chain. The term may encompass naturally, recombinantly, semi-synthetically or synthetically produced polypeptides. The term also encompasses polypeptides that carry one or more co- or post-expression-type modifications of the polypeptide chain, such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes polypeptide variants or mutants which carry amino acid sequence variations vis-à-vis a corresponding native polypeptide, such as, e.g., amino acid deletions, additions and/or substitutions. The term contemplates both full-length polypeptides and polypeptide parts or fragments, e.g., naturally-occurring polypeptide parts that ensue from processing of such full-length polypeptides.

The term “peptide” as used throughout this specification preferably refers to a polypeptide as used herein consisting essentially of 50 amino acids or less, e.g., 45 amino acids or less, preferably 40 amino acids or less, e.g., 35 amino acids or less, more preferably 30 amino acids or less, e.g., 25 or less, 20 or less, 15 or less, 10 or less or 5 or less amino acids.

A peptide, polypeptide or protein can be naturally occurring, e.g., present in or isolated from nature, e.g., produced or expressed natively or endogenously by a cell or tissue and optionally isolated therefrom. A peptide, polypeptide or protein can be recombinant, i.e., produced by recombinant DNA technology, and/or can be, partly or entirely, chemically or biochemically synthesised. Without limitation, a peptide, polypeptide or protein can be produced recombinantly by a suitable host or host cell expression system and optionally isolated therefrom (e.g., a suitable bacterial, yeast, fungal, plant or animal host or host cell expression system), or produced recombinantly by cell-free translation or cell-free transcription and translation, or non-biological peptide, polypeptide or protein synthesis.

The term “nucleic acid” as used herein typically refers to an oligomer or polymer (preferably a linear polymer) of any length composed essentially of nucleotides. A nucleotide unit commonly includes a heterocyclic base, a sugar group, and at least one, e.g. one, two, or three, phosphate groups, including modified or substituted phosphate groups. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups. Nucleic acids as intended herein may include naturally occurring nucleotides, modified nucleotides or mixtures thereof. A modified nucleotide may include a modified heterocyclic base, a modified sugar moiety, a modified phosphate group or a combination thereof. Modifications of phosphate groups or sugars may be introduced to improve stability, resistance to enzymatic degradation, or some other useful property. The term “nucleic acid” further preferably encompasses DNA, RNA and DNA/RNA hybrid molecules, specifically including hnRNA, pre-mRNA, mRNA, cDNA, genomic DNA, amplification products, oligonucleotides, and synthetic (e.g., chemically synthesised) DNA, RNA or DNA/RNA hybrids. A nucleic acid can be naturally occurring, e.g., present in or isolated from nature; or can be non-naturally occurring, e.g., recombinant, i.e., produced by recombinant DNA technology, and/or partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

The reference to any peptides, polypeptides, proteins or nucleic acids encompass such peptides, polypeptides, proteins or nucleic acids of any organism where found, and particularly of animals, preferably warm-blooded animals, more preferably vertebrates, yet more preferably mammals, including humans and non-human mammals, still more preferably of humans.

The term “peptidomimetic” as used herein refers to a non-peptide agent that is a topological analogue of a corresponding peptide. Methods of rationally designing peptidomimetics of peptides are known in the art. For example, by means of a guidance, the rational design of three peptidomimetics based on the sulphated 8-mer peptide CCK26-33, and of two peptidomimetics based on the 11-mer peptide Substance P, and related peptidomimetic design principles, are described in Horwell 1995 (Trends Biotechnol 13: 132-134).

The term “aptamer” as used herein refers to single-stranded or double-stranded oligo-DNA, oligo-RNA or oligo-DNA/RNA or any analogue thereof that can specifically bind to a target molecule. Advantageously, aptamers can display fairly high specificity and affinity (e.g., K_{A} in the order 1×10⁹ M⁻¹) for their targets. Aptamer production is described inter alia in U.S. Pat. No. 5,270,163; Ellington & Szostak 1990 (Nature 346: 818-822); Tuerk & Gold 1990 (Science 249: 505-510); or “The Aptamer Handbook: Functional Oligonucleotides and Their Applications”, by Klussmann, ed., Wiley-VCH 2006, ISBN 3527310592, incorporated by reference herein. The term also encompasses photoaptamers, i.e., aptamers that contain one or more photoreactive functional groups that can covalently bind to or crosslink with a target molecule.

The term “small organic molecule” or “small molecule” as used herein encompasses organic compounds with a size comparable to those organic molecules generally used in pharmaceuticals. The term excludes biological macromolecules (e.g., proteins, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da.

The term “antibody” is used herein in its broadest sense and generally refers to any immunologic binding agent, such as a whole antibody, including without limitation a chimeric, humanized, human, recombinant, transgenic, grafted and single chain antibody, and the like, or any fusion proteins, conjugates, fragments, or derivatives thereof that contain one or more domains that selectively bind to an antigen of interest. The term antibody thereby includes a whole immunoglobulin molecule, a monoclonal antibody, a chimeric antibody, a humanized antibody, a human antibody, or an immunologically effective fragment of any of these. The term thus specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest), as well as multivalent and/or multi-specific composites of such fragments. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro, in cell culture, or in vivo.

In certain embodiments, the specific ACKR2 modulator may be an antibody fragment.

The term “antibody fragment” or “antigen-binding moiety” comprises a portion or region of a full length antibody, generally the antigen binding or variable domain thereof. Examples of antibody fragments include Fab, Fab′, F(ab)2, Fv, scFv fragments, single domain (sd)Fv, such as V_{H} domains, V_{L} domains and V_{HH} domains, diabodies, linear antibodies, single-chain antibody molecules, in particular heavy-chain antibodies; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning.

In particular embodiments, the specific ACKR2 modulator may be a Nanobody®. The terms “Nanobody®” and “Nanobodies®” are trademarks of Ablynx NV (Belgium). The term “Nanobody” is well-known in the art and as used herein in its broadest sense encompasses an immunological binding agent obtained (1) by isolating the V_{HH} domain of a naturally occurring heavy-chain antibody, preferably a heavy-chain antibody derived from camelids; (2) by expression of a nucleotide sequence encoding a naturally occurring V_{HH} domain; (3) by “humanization” of a naturally occurring V_{HH} domain or by expression of a nucleic acid encoding a such humanized V_{HH} domain; (4) by “camelization” of a naturally occurring V_{H} domain from any animal species, and in particular from a mammalian species, such as from a human being, or by expression of a nucleic acid encoding such a camelized V_{H} domain; (5) by “camelisation” of a “domain antibody” or “dAb” as described in the art, or by expression of a nucleic acid encoding such a camelized dAb; (6) by using synthetic or semi-synthetic techniques for preparing proteins, polypeptides or other amino acid sequences known per se; (7) by preparing a nucleic acid encoding a Nanobody using techniques for nucleic acid synthesis known per se, followed by expression of the nucleic acid thus obtained; and/or (8) by any combination of one or more of the foregoing. “Camelids” as used herein comprise old world camelids (Camelus bactrianus and Camelus dromaderius) and new world camelids (for example Lama paccos, Lama glama and Lama vicugna).

In particular embodiments, the ACKR2 modulator as described herein is selected from a group consisting of an anti-ACKR2 antibody, an ACKR2-directed gene-editing system, an RNAi agent directed against ACKR2, a fragment or derivative of CCL2, a fragment or derivative of CCL3, a fragment or derivative of CCL4, a fragment or derivative of CCL5, a fragment or derivative of CCL7, a fragment or derivative of CCL8, a fragment or derivative of CCL11, a fragment or derivative of CCL13, a fragment or derivative of CCL14, a fragment or derivative of CCL17, a fragment or derivative of CCL22, a fragment or derivative of CXCL10 and any combination thereof.

In particular embodiments, the ACKR2 modulator as described herein is selected from a group consisting of an anti-ACKR2 antibody, an ACKR2-directed gene-editing system, an RNAi agent directed against ACKR2, a fragment or derivative of CCL5, a fragment or derivative of CXCL10, and any combination thereof.

In particular embodiments, the ACKR2 modulator as described herein is selected from a group consisting of an antibody specifically binding ACKR2, a gene-editing system specifically directed against ACKR2, an RNAi agent specifically directed against ACKR2, a fragment or derivative of CCL5, a fragment or derivative of CXCL10, and any combination thereof.

In particular embodiments, the ACKR2 modulator as described herein is selected from a group consisting of an anti-ACKR2 antibody, an ACKR2-directed gene-editing system, an RNAi agent directed against ACKR2, and any combination thereof.

In particular embodiments, the ACKR2 modulator as described herein is selected from a group consisting of an antibody specifically binding ACKR2, a gene-editing system specifically directed against ACKR2, an RNAi agent specifically directed against ACKR2, and any combination thereof

In particular embodiments, the ACKR2 modulator as described herein is selected from a group consisting of an antibody specifically binding ACKR2 and an RNAi agent specifically directed against ACKR2.

In particular embodiments, the ACKR2 modulator as described herein is selected from a group consisting of an antibody specifically binding ACKR2, siRNA specifically directed against ACKR2 and shRNA specifically directed against ACKR2.

In particular embodiments, the ACKR2 modulator as described herein is a polypeptide, protein or peptide.

In particular embodiments, the ACKR2 modulator is an antibody, such as anti-ACKR2 antibody. In particular embodiments, the ACKR2 modulator is an antibody specifically binding ACKR2.

In particular embodiments, the ACKR2 modulator is an anti-ACKR2 antibody, preferably an antibody specifically binding ACKR2, which reduces and/or prevents the interaction between ACKR2 and CCL5 and/or CXCL10 (e.g. which blocks the binding of CCL5 and/or CXCL10 to ACKR2).

In particular embodiments, the antibody specifically binding ACKR2 is an antibody specifically binding the N-terminal region of the ACKR2 protein, preferably the N-terminal region of the human ACKR2 protein (e.g. human ACKR2 protein sequence is annotated under NCBI Genbank accession number NP_001287.2, and Uniprot (www.uniprot.org) accession number O00590.2).

In particular embodiments, the antibody specifically binding ACKR2 is an antibody specifically binding the amino acid sequence MAATASPQPLATEDADSENSSFYYYDYLDEVAFMLCRKDAVVSFGKVFLP (SEQ ID NO: 1), amino acid sequence QTHENPKGVWNCHADFGGHGTIWKLFLRFQQNLL (SEQ ID NO: 2) or amino acid sequence LHTLLDLQVFGNCEVSQHLDYA (SEQ ID NO: 3).

For example, the ACKR2 modulator may be an anti-ACKR2 antibody with product number ab1656 from Abcam.

In particular embodiments, the ACKR2 modulator as described herein is a fragment or derivative of a ACKR2 ligand, preferably an ACKR2-specific ligand, such as CCL2, CCL3, CCL4, CCL5, CCL7, CCL8, CCL11, CCL13, CCL14, CCL17, CCL22 or CXCL10.

In particular embodiments, the ACKR2 modulator as described herein is selected from a group consisting of a fragment or derivative of CCL2 (e.g. with UniProt accession number P13500), a fragment or derivative of CCL3 (e.g. with UniProt accession number P10147), a fragment or derivative of CCL4 (e.g. with UniProt accession number P13236), a fragment or derivative of CCL5 (e.g. with UniProt accession number P13501), a fragment or derivative of CCL7 (e.g. with UniProt accession number P80098), a fragment or derivative of CCL8 (e.g. with UniProt accession number P80075), a fragment or derivative of CCL11 (e.g. with UniProt accession number P51671, a fragment or derivative of CCL13 (e.g. with UniProt accession number Q99616), a fragment or derivative of CCL14 (e.g. with UniProt accession number Q16627), a fragment or derivative of CCL17 (e.g. with UniProt accession number Q92583), a fragment or derivative of CCL22 (e.g. with UniProt accession number O00626), a fragment or derivative of CXCL10 (e.g. with UniProt accession number P02778) and any combination thereof.

In particular embodiments, the ACKR2 modulator as described herein comprises, consists essentially of, or consists of a fragment or derivative of CCL5 or CXCL10, preferably a fragment or derivative of CXCL10, more preferably a fragment or derivative of human CXCL10.

In particular embodiments, the ACKR2 modulator as described herein comprises, consists essentially of, or consists of a fragment or derivative of human CCL5 (e.g. with UniProt accession number P13501) and/or CXCL10 (e.g. with UniProt accession number P02778).

In preferred embodiments, the ACKR2 modulator as described herein comprises, consists essentially of, or consists of a fragment or derivative of CCL5 and CXCL10.

The term “fragment” with reference to a peptide, polypeptide, or protein generally denotes a N- and/or C-terminally truncated form of the peptide, polypeptide, or protein. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the amino acid sequence length of said peptide, polypeptide, or protein. For example, insofar not exceeding the length of the full-length peptide, polypeptide, or protein, a fragment may include a sequence of 5 consecutive amino acids, or 10 consecutive amino acids, or 20 consecutive amino acids, or 30 consecutive amino acids, e.g., consecutive amino acids, such as for example 50 consecutive amino acids, e.g., 60, 70, 80, 90, consecutive amino acids of the corresponding full-length peptide, polypeptide, or protein.

The term “derivative” of a protein, polypeptide or peptide refers proteins, polypeptides or peptides the sequence (i.e., amino acid sequence) of which is substantially identical (i.e., largely but not wholly identical) to the sequence of said protein or polypeptide, or fragment thereof, e.g., at least about 80% identical or at least about 85% identical, e.g., preferably at least about 90% identical, e.g., at least 91% identical, 92% identical, more preferably at least about 93% identical, e.g., at least 94% identical, even more preferably at least about 95% identical, e.g., at least 96% identical. Protein, polypeptide or peptide derivative includes proteins, polypeptides or peptides which carry amino acid sequence variations vis-à-vis a corresponding native protein, polypeptide or peptide, such as, e.g., amino acid deletions, additions and/or substitutions. The term contemplates both full-length proteins, polypeptides and peptides and polypeptides and protein, polypeptide and peptide parts or fragments. Preferably, the derivative retains at least about 85%, more preferably at least 90%, even more preferably at least 95% of the features of interest of the protein, polypeptide or peptide, though it will be understood that some features may be modified. In the context of the present invention, a derivative of a given protein, polypeptide and peptide may retain at least about 85%, more preferably at least 90%, even more preferably at least 95% of the binding activity of the protein, polypeptide and peptide to ACKR2. In further particular embodiments, the binding of the derivative to ACKR2 may be improved.

In particular embodiments, the ACKR2 modulator as described herein comprises, consists essentially of, or consists of a fragment and/or derivative of the N terminus of CCL5 and/or CXCL10, preferably human CCL5 (e.g. with UniProt accession number P13501) and/or CXCL10 (e.g. with UniProt accession number P02778). In preferred embodiments, the ACKR2 modulator as described herein comprises, consists essentially of, or consists of the one or more amino acid domain(s) of CCL5 and/or CXCL10 required for binding to the ACKR2 orthosteric binding pocket and/or to the conserved tyrosine motif at the N terminus of ACKR2, for example the conserved tyrosine motif at the N terminus of ACKR2 as described in Hewit et al., The N-terminal Region of the Atypical Chemokine Receptor ACKR2 Is a Key Determinant of Ligand Binding. J Biol. Chem. 2014. 289(18):12330-12342.

In particular embodiments, the ACKR2 modulator as described herein specifically binds to ACKR2.

In particular embodiments, the ACKR2 modulator as described herein specifically binds to the ACKR2 polypeptide (i.e. the ACKR2 receptor), the ACKR2 gene (e.g. CRISPR guide RNA directed against ACKR2) or the ACKR2 mRNA (e.g. siRNA directed against ACKR2).

In particular embodiments, the ACKR2 modulator as described herein is a binding agent which specifically binds to ACKR2 preventing the binding of CCL5 and/or CXCL10.

In particular embodiments, the ACKR2 modulator as described herein specifically binds to the ACKR2 receptor. Such a binding agent may be an Antibody. The antibody may be a polyclonal antibody, a monoclonal antibody, a chimeric antibody, a humanized antibody, a primatized antibody, a human antibody, a Nanobody or fragments thereof, or other similar types of antibodies or antibody fragments described in the art. Alternative binding agents which can be developed to specifically interfere with the binding site of CCL5 and/or CXCL10 to ACKR2 include but are not limited to Alphabodies, Affibody molecules, Affilins, Affimers, Anticalines, Avimers, DARPins, Fynomers, Kunitz domain peptides, Monobodies and NanoCLAMPS.

It is also envisaged that the ACKR2 modulator in particular embodiments is a combination of different binding agents. For instance, in particular embodiments, the ACKR2 modulator as described herein is a mixture of one or more of the group consisting of a polyclonal antibody, a monoclonal antibody, a chimeric antibody, a humanized antibody, a primatized antibody, a human antibody and a Nanobody.

In more particular embodiments, wherein the ACKR2 modulator is a polypeptide, protein or peptide, the ACKR2 modulator may be coupled to an agent selected from the group consisting of a detectable label such as a fluorescent protein or enzyme, an immunoglobulin Fc region (e.g. IgG2a Fc), protoxin, toxin or pharmaceutical, preferably a fluorescent protein, an enzyme, an immunoglobulin Fc region, a protoxin or a toxin.

The ACKR2 modulator may inhibit binding of CCL5 and/or CXCL10 to ACKR2 by specifically preventing expression of ACKR2. Several types of molecules are known which are capable of reducing expression of a target compound. In particular embodiments, the ACKR2 modulator is a gene-editing system, an antisense agent, an RNAi agent, such as siRNA or shRNA.

In particular embodiments, the ACKR2 modulator is an ACKR2-directed gene-editing system, an antisense agent directed against ACKR2, an RNAi agent directed against ACKR2, such as siRNA directed against ACKR2 or shRNA s directed against ACKR2. In particular embodiments, the gene editing system is specifically directed against ACKR2. In particular embodiments, the antisense agent is specifically directed against ACKR2. In particular embodiments, the RNAi agent is specifically directed against ACKR2.

In particular embodiments, such as wherein the specific ACKR2 modulator decreases the quantity and/or expression level of ACKR2, the ACKR2 modulator is a (endo)nuclease or a variant thereof having altered or modified activity. In particular embodiments, the ACKR2 modulator is a Clustered Regularly Interspaced Short Palindromic Repeats (CRISPR) associated (Cas) (endo)nuclease system (i.e. endonuclease and targeting RNA combination), more particularly a CRISPR system comprising an endonuclease such as Cas9, Cpf1, or C2c2. In further embodiments, the ACKR2 modulator is a zinc finger nuclease (ZFN), a transcription factor-like effector nuclease (TALEN), a meganuclease, or modifications thereof.

Targeted genome modification is a powerful tool for genetic manipulation of cells and organisms, including mammals. Genome modification or gene editing, including insertion, deletion or replacement of DNA in the genome, can be carried out using a variety of known gene editing systems. The term “gene editing system” or “genome editing system” as used herein refers to a tool to induce one or more nucleic acid modifications, such as DNA or RNA modifications, into a specific DNA or RNA sequence within a cell. Gene editing systems typically make use of an agent capable of inducing a nucleic acid modification. In certain embodiments, the agent capable of inducing a nucleic acid modification may be a (endo)nuclease or a variant thereof having altered or modified activity. In particular embodiments, the (endo)nuclease comprises programmable, sequence-specific DNA- or RNA-binding modules linked to a nonspecific DNA or RNA cleavage domain. Alternatively, they are guided to a specific target by an interacting targeting RNA molecule, which is sequence-specific. In DNA, the nucleases create site-specific double-strand breaks at desired locations in the genome. The induced double-stranded breaks are repaired through nonhomologous end-joining or homologous recombination, resulting in targeted mutations. In certain embodiments, said (endo)nuclease may be RNA-guided. In certain embodiments, said (endo)nuclease can be engineered nuclease such as a CRISPR associated (Cas) (endo)nuclease, such as Cas9, Cpf1, or C2c2, a ZFN, a TALEN, a meganuclease, or modifications thereof. Methods for using TALEN technology, Zinc Finger technology and CRISPR/Cas technology are known by the skilled person.

In particular embodiments, the ACKR2 modulator as described herein may be a CRISPR system comprising a CRISPR/Cas enzyme and one or more targeting RNAs directed to the coding sequence encoding ACKR2.

Kits comprising the means for modulating, such as inhibiting or increasing, expression or activity of said ACKR2 by use of a gene editing system are also provided.

In particular embodiments, the specific ACKR2 modulator as disclosed herein is an antisense agents capable of binding to (annealing with) a sequence region in pre-mRNA or mRNA sequence of ACKR2. Particularly intended may be such RNAi agents configured to target mRNA of ACKR2.

The term “antisense” generally refers to an agent (e.g., an oligonucleotide as defined elsewhere in the specification) configured to specifically anneal with (hybridise to) a given sequence in a target nucleic acid, such as for example in a target DNA, hnRNA, pre-mRNA or mRNA, and typically comprises, consist essentially of or consist of a nucleic acid sequence that is complementary or substantially complementary to said target nucleic acid sequence. Antisense agents suitable for use herein may typically be capable of annealing with (hybridising to) the respective target nucleic acid sequences at high stringency conditions, and capable of hybridising specifically to the target under physiological conditions.

Preferably, to ensure specificity of antisense agents towards the desired target over unrelated molecules, the sequence of said antisense agents may be at least about 80% identical, preferably at least about 90% identical, more preferably at least about 95% identical, such as, e.g., about 96%, about 97%, about 98%, about 99% and up to 100% identical to the respective target sequence.

Antisense agents as intended herein preferably comprise or denote antisense molecules such as more preferably antisense nucleic acid molecules or antisense nucleic acid analogue molecules. Preferably, antisense agents may refer to antisense oligonucleotides or antisense oligonucleotide analogues.

The term “RNA interference agent” or “RNAi agent” refers to ribonucleic acid sequences, modified ribonucleic acid sequences, or DNA sequences encoding said ribonucleic acid sequences, which cause RNA interference and thus decrease expression of the target gene.

An RNAi (RNA interference) agent typically comprises, consists essentially of or consists of a double-stranded portion or region (notwithstanding the optional and potentially preferred presence of single-stranded overhangs) of annealed complementary strands, one of which has a sequence corresponding to a target nucleotide sequence (hence, to at least a portion of an mRNA) of the target gene to be down-regulated. The other strand of the RNAi agent is complementary to said target nucleotide sequence. Non-limiting examples of RNAi agents are shRNAs, siRNAs, miRNAs, and DNA-RNA hybrids.

Whereas the sequence of an RNAi agent need not be completely identical to a target sequence to be down-regulated, the number of mismatches between a target sequence and a nucleotide sequence of the RNAi agent is preferably no more than 1 in 5 bases, or 1 in 10 bases, or 1 in 20 bases, or 1 in 50 bases.

Preferably, to ensure specificity of RNAi agents towards the desired target over unrelated molecules, the sequence of said RNAi agents may be at least about 80% identical, preferably at least about 90% identical, more preferably at least about 95% identical, such as, e.g., about 96%, about 97%, about 98%, about 99% and up to 100% identical to the respective target sequence.

In particular embodiments, the ACKR2 modulator as described herein is an RNAi agent of between about 15 and about 60 nucleotides in length and which comprises a nucleotide sequence that is at least 70% identical to a region of the gene encoding ACKR2.

An RNAi agent may be formed by separate sense and antisense strands or, alternatively, by a common strand providing for fold-back stem-loop or hairpin design where the two annealed strands of an RNAi agent are covalently linked.

An siRNA molecule may be typically produced, e.g., synthesised, as a double stranded molecule of separate, substantially complementary strands, wherein each strand is about 18 to about 35 bases long, preferably about 19 to about 30 bases, more preferably about 20 to about 25 bases and even more preferably about 21 to about 23 bases.

shRNA is in the form of a hairpin structure. shRNA can be synthesized exogenously or can be formed by transcribing from RNA polymerase III promoters in vivo. Preferably, shRNAs can be engineered in host cells or organisms to ensure continuous and stable suppression of a desired gene. It is known that siRNA can be produced by processing a hairpin RNA in cells.

RNAi agents as intended herein may include any modifications as set out herein for nucleic acids and oligonucleotides, in order to improve their therapeutic properties such as a 3′ overhang.

RNAi agents as intended herein may be selected from a group comprising or consisting of short interfering nucleic acids and short interfering nucleic acid analogues (siNA) such as short interfering RNA and short interfering RNA analogues (siRNA), double-stranded RNA and double-stranded RNA analogues (dsRNA), micro-RNA and micro-RNA analogues (miRNA), and short hairpin RNA and short hairpin RNA analogues (shRNA).

Production of antisense agents and RNAi agents can be carried out by any processes known in the art, such as inter alia partly or entirely by chemical synthesis (e.g., routinely known solid phase synthesis; an exemplary an non-limiting method for synthesising oligonucleotides on a modified solid support is described in U.S. Pat. No. 4,458,066; in another example, diethyl-phosphoramidites are used as starting materials and may be synthesised as described by Beaucage et al. 1981 (Tetrahedron Letters 22: 1859-1862)), or partly or entirely by biochemical (enzymatic) synthesis, e.g., by in vitro transcription from a nucleic acid construct (template) using a suitable polymerase such as a T7 or SP6 RNA polymerase, or by recombinant nucleic acid techniques, e.g., expression from a vector in a host cell or host organism. Nucleotide analogues can be introduced by in vitro chemical or biochemical synthesis. In an embodiment, the antisense agents of the invention are synthesised in vitro and do not include antisense compositions of biological origin, or genetic vector constructs designed to direct the in vivo synthesis of antisense molecules.

In particular embodiments, the ACKR2 modulator as described herein may be an siRNA or shRNA directed against ACKR2. For example, a human ACKR2 silencer of ThermoFisher Scientific with Assay ID No. 145743 or shRNA of Sigma-Aldrich with product number SHCLNG-NM_021609 or SHCLND-NM_021609.

The specific ACKR2 modulator as disclosed herein may be an expressible molecule such as an antibody or a fragment or derivative thereof, a protein or polypeptide, a peptide, a nucleic acid, an antisense agent or an RNAi agent, it shall be understood that the ACKR2 modulator itself may be introduced to a subject or may be introduced by means of a recombinant nucleic acid comprising a sequence encoding the modulator operably linked to one or more regulatory sequences allowing for expression of said sequence encoding the agent (e.g., gene therapy or cell therapy).

Hence, the specific ACKR2 modulator may comprise a recombinant nucleic acid comprising a sequence encoding one or more desired proteins, polypeptides, peptides, antisense agents or RNAi agents, operably linked to one or more regulatory sequences allowing for expression of said sequence or sequences encoding the proteins, polypeptides, peptides, antisense agents or RNAi agents, e.g., in vitro, in a host cell, host organ and/or host organism (expression constructs). Such recombinant nucleic acid may be comprised in a suitable vector.

Accordingly, a further aspect provides a nucleic acid encoding the specific ACKR2 modulator.

By “encoding” is meant that a nucleic acid sequence or part(s) thereof corresponds, by virtue of the genetic code of an organism in question to a particular amino acid sequence, e.g., the amino acid sequence of one or more desired proteins or polypeptides, or to another nucleic acid sequence in a template-transcription product (e.g. RNA or RNA analogue) relationship.

A further aspect provides a nucleic acid expression cassette comprising the specific ACKR2 modulator or the nucleic acid encoding the specific ACKR2 modulator as described herein, operably linked to a promoter and/or transcriptional and translational regulatory signals.

The term “nucleic acid expression cassettes” as used herein refers to nucleic acid molecules, typically DNA, to which nucleic acid fragments may be inserted to be expressed, wherein said nucleic acid molecules comprise one or more nucleic acid sequences controlling the expression of the nucleic acid fragments. Non-limiting examples of such more nucleic acid sequences controlling the expression of the nucleic acid fragments include promoter sequences, open reading frames and transcription terminators.

Preferably, the nucleic acid expression cassette may comprise one or more open reading frames (ORF) encoding said one or more proteins, polypeptides or peptides. An “open reading frame” or “ORF” refers to a succession of coding nucleotide triplets (codons) starting with a translation initiation codon and closing with a translation termination codon known per se, and not containing any internal in-frame translation termination codon, and potentially capable of encoding a protein, polypeptide or peptide. Hence, the term may be synonymous with “coding sequence” as used in the art.

An “operable linkage” is a linkage in which regulatory sequences and sequences sought to be expressed are connected in such a way as to permit said expression. For example, sequences, such as, e.g., a promoter and an ORF, may be said to be operably linked if the nature of the linkage between said sequences does not: (1) result in the introduction of a frame-shift mutation, (2) interfere with the ability of the promoter to direct the transcription of the ORF, (3) interfere with the ability of the ORF to be transcribed from the promoter sequence. Hence, “operably linked” may mean incorporated into a genetic construct so that expression control sequences, such as a promoter, effectively control transcription/expression of a sequence of interest.

The precise nature of transcriptional and translational regulatory sequences or elements required for expression may vary between expression environments, but typically include a transcription terminator, and optionally an enhancer.

Reference to a “promoter” is to be taken in its broadest context and includes transcriptional regulatory sequences required for accurate transcription initiation and where applicable accurate spatial and/or temporal control of gene expression or its response to, e.g., internal or external (e.g., exogenous) stimuli. More particularly, “promoter” may depict a region on a nucleic acid molecule, preferably DNA molecule, to which an RNA polymerase binds and initiates transcription. A promoter is preferably, but not necessarily, positioned upstream, i.e., 5′, of the sequence the transcription of which it controls. Typically, in prokaryotes a promoter region may contain both the promoter per se and sequences which, when transcribed into RNA, will signal the initiation of protein synthesis (e.g., Shine-Dalgarno sequence). A promoter sequence can also include “enhancer regions”, which are one or more regions of DNA that can be bound with proteins (namely the trans-acting factors) to enhance transcription levels of genes in a gene-cluster. The enhancer, while typically at the 5′ end of a coding region, can also be separate from a promoter sequence, e.g., can be within an intronic region of a gene or 3′ to the coding region of the gene.

In embodiments, promoters contemplated herein may be constitutive or inducible. A constitutive promoter is understood to be a promoter whose expression is constant under the standard culturing conditions. Inducible promoters are promoters that are responsive to one or more induction cues. For example, an inducible promoter can be chemically regulated (e.g., a promoter whose transcriptional activity is regulated by the presence or absence of a chemical inducing agent such as an alcohol, tetracycline, a steroid, a metal, or other small molecule) or physically regulated (e.g., a promoter whose transcriptional activity is regulated by the presence or absence of a physical inducer such as light or high or low temperatures). An inducible promoter can also be indirectly regulated by one or more transcription factors that are themselves directly regulated by chemical or physical cues. Non-limiting examples of promoters include T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter.

In particular embodiments, the promoter is a cancer- or tumor-specific promoter. Non-limiting examples of cancer-specific promoters include ran, brms1 and mcm5 promoters. Non-limiting examples of tumor-specific promoters include E2F-1 promoter, HE4 promoter, LP promoter, and COX-2 promoter.

In particular embodiments, the promoter is a melanoma-specific promoter, such as the tyrosinase promoter as described in Lillehammer T. et al., Melanoma-specific expression in first-generation adenoviral vectors in vitro and in vivo—use of the human tyrosinase promoter with human enhancers, Cancer Gene Ther. 2005, 12(11):864-72.

The terms “terminator” or “transcription terminator” refer generally to a sequence element at the end of a transcriptional unit which signals termination of transcription. For example, a terminator is usually positioned downstream of, i.e., 3′ of ORF(s) encoding a polypeptide of interest. For instance, where a recombinant nucleic acid contains two or more ORFs, e.g., successively ordered and forming together a multi-cistronic transcription unit, a transcription terminator may be advantageously positioned 3′ to the most downstream ORF.

In particular embodiments, the nucleic acid expression cassette comprises the specific ACKR2 modulator or the nucleic acid encoding the specific ACKR2 modulator as disclosed herein, operably linked to one or more promoters, enhancers, ORFs and/or transcription terminators.

A further aspect provides a vector comprising the specific ACKR2 modulator, the nucleic acid encoding the specific ACKR2 modulator or the nucleic acid expression cassette as described herein, such as a viral vector.

The term “vector” or “expression vector” as used in the application refers to nucleic acid molecules, e.g. double-stranded DNA, which may have inserted into it another nucleic acid molecule (the insert nucleic acid molecule) such as, but not limited to, a cDNA molecule. The vector is used to transport the insert nucleic acid molecule into a suitable host cell. A vector may contain the necessary elements that permit transcribing the insert nucleic acid molecule, and, optionally, translating the transcript into a peptide, protein or polypeptide. The insert nucleic acid molecule may be derived from the host cell, or may be derived from a different cell or organism. Once in the host cell, the vector can replicate independently of, or coincidental with, the host chromosomal DNA, and several copies of the vector and its inserted nucleic acid molecule may be generated. The vectors can be episomal vectors (i.e., that do not integrate into the genome of a host cell), or can be vectors that integrate into the host cell genome. The term “vector” may thus also be defined as a gene delivery vehicle that facilitates gene transfer into a target cell. This definition includes both non-viral and viral vectors. Non-viral vectors include but are not limited to cationic lipids, liposomes, nanoparticles, PEG, PEI, plasmid vectors (e.g. pUC vectors, bluescript vectors (pBS) and pBR322 or derivatives thereof that are devoid of bacterial sequences (minicircles)) transposons-based vectors (e.g. PiggyBac (PB) vectors or Sleeping Beauty (SB) vectors), etc. Viral vectors are derived from viruses and include but are not limited to retroviral, lentiviral, adeno-associated viral, adenoviral, herpes viral, hepatitis viral vectors or the like. Typically, but not necessarily, viral vectors are replication-deficient as they have lost the ability to propagate in a given cell since viral genes essential for replication have been eliminated from the viral vector. However, some viral vectors can also be adapted to replicate specifically in a given cell, such as e.g. a cancer cell, and are typically used to trigger the (cancer) cell-specific (onco)lysis. Virosomes are a non-limiting example of a vector that comprises both viral and non-viral elements, in particular they combine liposomes with an inactivated HIV or influenza virus (Yamada et al., 2003). Another example encompasses viral vectors mixed with cationic lipids.

The specific ACKR2 modulator as described herein may be suitably obtained through expression by host cells or host organisms, transformed with an expression construct encoding and configured for expression of said specific ACKR2 modulator in said host cells or host organisms, followed by purification of the specific ACKR2 modulator.

Hence, a further aspect provides a host cell comprising the nucleic acid, nucleic acid expression cassette or vector as described herein.

In certain embodiments, the host cell may be a bacterial cell, a yeast cell, an animal cell, or a mammalian cell.

The terms “host cell” and “host organism” may suitably refer to cells or organisms encompassing both prokaryotes, such as bacteria, and eukaryotes, such as yeast, fungi, protozoan, plants and animals.

Contemplated as host cells are inter alia unicellular organisms, such as bacteria (e.g., E. coli, Salmonella tymphimurium, Serratia marcescens, or Bacillus subtilis), yeast (e.g., Saccharomyces cerevisiae or Pichia pastoris), (cultured) plant cells (e.g., from Arabidopsis thaliana or Nicotiana tobaccum) and (cultured) animal cells (e.g., vertebrate animal cells, mammalian cells, primate cells, human cells or insect cells). Contemplated as host organisms are inter alia multi-cellular organisms, such as plants and animals, preferably animals, more preferably warm-blooded animals, even more preferably vertebrate animals, still more preferably mammals, yet more preferably primates; particularly contemplated are such animals and animal categories which are non-human.

The ACKR2 modulator as described herein may be suitably isolated and/or purified. Purified nucleic acids, proteins, polypeptides or peptides may be obtained by known methods including, for example, laboratory or recombinant synthesis, chromatography, preparative electrophoresis, centrifugation, precipitation, affinity purification, etc.

In particular embodiments, the vector comprising the nucleic acid as described herein is a viral vector, preferably a viral vector specifically directed towards cancer, preferably specifically directed towards melanoma or colorectal cancer. Melanoma-specific targeting of a viral vector may be achieved, for example, by fusion of a single chain antibody recognizing the high molecular-weight melanoma-associated antigen (HMWMAA), followed by a blocking peptide and a matrix metalloprotease cleavage site, to the amino terminus of the murine leukemia virus amphotropic strain envelope as described by Martin et al., Envelope-Targeted Retrovirus Vectors Transduce Melanoma Xenografts but Not Spleen or Liver, Molecular Therapy. 2002, 5(3): 269-274.

In particular embodiments, the nucleic acid encoding the agent as taught herein may be comprised within a vector providing for a signal peptide. The signal peptide may be a homologous or heterologous signal peptide, depending on the host cell used for production of the ACKR2 modulator as described herein. Furthermore, for prokaryotic expression of the ACKR2 modulator as described herein, a protease cleavage site motif may be present C-terminally of said signal peptide and N-terminally of the ACKR2 modulator as described herein.

A further aspect provides a pharmaceutical composition comprising the specific ACKR2 modulator as described herein, the nucleic acid encoding the specific ACKR2 modulator, the nucleic acid expression cassette as described herein or the vector as described herein, and optionally a pharmaceutically acceptable carrier.

The term “pharmaceutically acceptable” as used herein is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, antioxidants, tonicity controlling agents, absorption delaying agents, and the like. The use of such media and agents for pharmaceutical active substances is well known in the art. Except insofar as any conventional media or agent is incompatible with the active substance, its use in the therapeutic compositions may be contemplated.

Illustrative, non-limiting carriers for use in formulating the pharmaceutical compositions include, for example, oil-in-water or water-in-oil emulsions, aqueous compositions with or without inclusion of organic co-solvents suitable for intravenous (IV) use, liposomes or surfactant-containing vesicles, microspheres, microbeads and microsomes, powders, tablets, capsules, suppositories, aqueous suspensions, aerosols, and other carriers apparent to one of ordinary skill in the art.

Pharmaceutical compositions as intended herein may be formulated for essentially any route of administration, such as without limitation, oral administration (such as, e.g., oral ingestion or inhalation), intranasal administration (such as, e.g., intranasal inhalation or intranasal mucosal application), parenteral administration (such as, e.g., subcutaneous, intravenous (I.V.), intramuscular, intraperitoneal or intrasternal injection or infusion), transdermal or transmucosal (such as, e.g., oral, sublingual, intranasal) administration, topical administration, rectal, vaginal or intra-tracheal instillation, and the like. In this way, the therapeutic effects attainable by the methods and compositions can be, for example, systemic, local, tissue-specific, etc., depending of the specific needs of a given application.

In particular embodiments, the compositions of the present invention are designed for tissue-specific delivery. Indeed, it can be of interest to limit the delivery of the ACKR2 modulator to the environment of the tumor, in order to (a) optimize the effect of the modulator and (b) limit toxicity. Tissue-specificity can be achieved at different levels and will be dependent on the nature of the ACKR2 modulator. To some extent, tissue-specificity can be achieved by local delivery of the compositions of the invention. Tissue-specific vectors and promotors are known in the art. Where the ACKR2 modulator is a protein, peptide or small-molecule, targeting can be achieved through binding with a targeting agent such as an antibody.

The dosage or amount of the specific ACKR2 modulator as taught herein, optionally in combination with one or more other active compounds to be administered, depends on the individual case and is, as is customary, to be adapted to the individual circumstances to achieve an optimum effect. Thus, the unit dose and regimen depend on the nature and the severity of the disorder to be treated, and also on factors such as the species of the subject, the sex, age, body weight, general health, diet, mode and time of administration, immune status, and individual responsiveness of the human or animal to be treated, efficacy, metabolic stability and duration of action of the compounds used, on whether the therapy is acute or chronic or prophylactic, or on whether other active compounds are administered in addition to the agent of the invention. In order to optimize therapeutic efficacy, the ACKR2 modulator, the nucleic acid encoding the ACKR2 modulator, the nucleic acid expression cassette, the vector, the host cell or the pharmaceutical composition as described or taught herein can be first administered at different dosing regimens. Typically, levels of the agent in a tissue can be monitored using appropriate screening assays as part of a clinical testing procedure, e.g., to determine the efficacy of a given treatment regimen. The frequency of dosing is within the skills and clinical judgement of medical practitioners (e.g., doctors, veterinarians or nurses). Typically, the administration regime is established by clinical trials which may establish optimal administration parameters. However, the practitioner may vary such administration regimes according to the one or more of the aforementioned factors, e.g., subject's age, health, weight, sex and medical status. The frequency of dosing can be varied depending on whether the treatment is prophylactic or therapeutic.

Toxicity and therapeutic efficacy of the agent as described herein or pharmaceutical compositions comprising the same can be determined by known pharmaceutical procedures in, for example, cell cultures or experimental animals. These procedures can be used, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Pharmaceutical compositions that exhibit high therapeutic indices are preferred. While pharmaceutical compositions that exhibit toxic side effects can be used, care should be taken to design a delivery system that targets such compounds to the site of affected tissue in order to minimize potential damage to normal cells (e.g., non-target cells) and, thereby, reduce side effects.

The data obtained from the cell culture assays and animal studies can be used in formulating a range of dosage for use in appropriate subjects. The dosage of such pharmaceutical compositions lies generally within a range of circulating concentrations that include the ED50 with little or no toxicity. The dosage may vary within this range depending upon the dosage form employed and the route of administration utilized. For a pharmaceutical composition used as described herein, the therapeutically effective dose can be estimated initially from cell culture assays. A dose can be formulated in animal models to achieve a circulating plasma concentration range that includes the IC50 (i.e., the concentration of the pharmaceutical composition which achieves a half-maximal inhibition of symptoms) as determined in cell culture. Such information can be used to more accurately determine useful doses in humans. Levels in plasma can be measured, for example, by high performance liquid chromatography.

As described elsewhere herein, the specific ACKR2 modulator can be used to improve the response of a subject to anticancer immunotherapy, such as to an immune checkpoint modulator, preferably an immune checkpoint inhibitor. Accordingly, in particular embodiments, the invention relates to the combined treatment of a patient with the specific ACKR2 modulator of the invention and anticancer immunotherapy, such as a checkpoint modulator. Thus, in particular embodiments, the pharmaceutical composition as taught herein further comprises anticancer immunotherapy, preferably one or more immune checkpoint modulators, more preferably one or more immune checkpoint inhibitors. Immune checkpoint modulators and immune checkpoint inhibitors are known in the art and are described elsewhere herein.

In particular embodiments, the pharmaceutical composition as taught herein further comprises one or more (such as one, two, three or four) anticancer immunotherapies selected from the group consisting of an immune checkpoint modulator (e.g. immune checkpoint inhibitor), a monoclonal antibody (e.g. alemtuzumab or trastuzumab), chimeric antigen receptor T cells, an oncolytic virus, a cancer vaccine, a STING agonist, autologous or heterologous tumor-infiltrating lymphocytes, and any combination thereof.

In particular embodiments, the pharmaceutical composition as taught herein further comprises one or more (such as one, two, three or four) anticancer immunotherapies selected from the group consisting of an immune checkpoint inhibitor (such as a CD3-targeted bispecific antibody, anti-NKG2A antibody, anti-KIR antibody, anti-CD47 antibody, anti-CD24 antibody, anti-CD73 antibody, anti-TNFR2 antibody or anti-SIRPα antibody), a monoclonal antibody (e.g. alemtuzumab or trastuzumab), chimeric antigen receptor T cells, an oncolytic virus, a cancer vaccine, a STING agonist, autologous or heterologous tumor-infiltrating lymphocytes, and any combination thereof. In particular embodiments, the pharmaceutical composition as taught herein further comprises one or more immune checkpoint modulators selected from the group consisting of CTLA-4 inhibitors, PD-1 inhibitors, PD-L1 inhibitors, CD3 inhibitors, CD24 inhibitors, CD73 inhibitors, ICOS agonists, OX40 agonists, glucocorticoid-induced TNF receptor-related gene (GITR) agonists, CD137 agonists, CD40 agonists, CD27 agonists, CD70 agonists, lymphocyte activation gene 3 (LAG-3) inhibitors, T-cell immunoglobulin- and mucin-domain-containing molecule 3 (TIM-3) inhibitors, T cell immunoglobulin and ITIM domain (TIGIT) inhibitors, V-domain Ig suppressor of T-cell activation (VISTA) inhibitors, CD47 inhibitors, anti-SIRPα inhibitors, Indoleamine-2,3-dioxygenase (IDO) inhibitors, CD94 inhibitors, NKG2A inhibitors, killer immunoglobulin-like receptor (KIR) inhibitors, CD96 inhibitors, TNFR2 inhibitors, and any combination thereof.

In particular embodiments, the pharmaceutical composition as taught herein further comprises one or more immune checkpoint inhibitors selected from the group consisting of CTLA-4 inhibitors, PD-1 inhibitors, PD-L 1 inhibitors, CD3 inhibitors, CD24 inhibitors, CD73 inhibitors, lymphocyte activation gene 3 (LAG-3) inhibitors, T-cell immunoglobulin- and mucin-domain-containing molecule 3 (TIM-3) inhibitors, T cell immunoglobulin and ITIM domain (TIGIT) inhibitors, V-domain Ig suppressor of T-cell activation (VISTA) inhibitors, CD47 inhibitors, anti-SIRPα inhibitors, Indoleamine-2,3-dioxygenase (IDO) inhibitors, CD94 inhibitors, NKG2A inhibitors, killer immunoglobulin-like receptor (KIR) inhibitors, CD96 inhibitors, TNFR2 inhibitors, and any combination thereof.

In particular embodiments, the pharmaceutical composition as taught herein further comprises one or more (such as one, two, three or four) immune modulators, preferably one or more immune checkpoint inhibitors, selected from the group consisting of ipilimumab, tremelimumab, nivolumab, pembrolizumab, pidilizumab, MEDI0680, atezolizumab, BMS-936559, avelumab (MSB0010718C), durvulamab (MEDI4736), cemiplimab, tislelizumab, sintilimab, JTX-2011, JTX-4014, MEDI-570, GSK3359609, MEDI6469, MEDI6383, MEDI0562, PF-04518660, INCAGN01949, MOXR0916, GSK3174998, TRX518, MEDI1873, GWN323, MK-4166, INCAGN01876, OMP-336B11, PF-05082567, IMP321, LAG525, BMS986016, REGN3767, TSR-033, MGD013, TSR-022, LY3321367, MBG453, OMP-313M32, MTIG7192A/RG6058, JNJ-610588, CA-170, Hu5F9-G4, TTI-621, CC-90002, ALX148, Epacadostat (INCB024360), Pf-06840003, GDC-0919, NLG802, IPH2201 (monalizumab), BMS-986015/IPH-2102 (lirilumab), IPH4102, IPH2101, anti-CD73 antibody from innate pharma with identifier IPH5301, MEDI9447 (oleclumab), CPI-006, TJ004309, BMS-986179, BI-1808, BI-1910 agonist, and any combination thereof.

In particular embodiments, the pharmaceutical composition as taught herein further comprises one or more immune checkpoint inhibitors selected from the group consisting of ipilimumab, tremelimumab, nivolumab, pembrolizumab, pidilizumab, MEDI0680, atezolizumab, BMS-936559, avelumab (MSB0010718C), durvulamab (MEDI4736), Cemiplimab, Tislelizumab, Sintilimab, JTX-4014, IMP321, LAG525, BMS986016, REGN3767, TSR-033, MGD013, TSR-022, LY3321367, MBG453, OMP-313M32, MTIG7192A/RG6058, JNJ-610588, CA-170, Hu5F9-G4, TTI-621, CC-90002, ALX148, Epacadostat (INCB024360), Pf-06840003, GDC-0919, NLG802, IPH2201 (monalizumab), BMS-986015/IPH-2102 (lirilumab), IPH4102, anti-CD73 antibody from innate pharma with identifier IPH5301, MEDI9447 (oleclumab), CPI-006, TJ004309, BMS-986179, BI-1808, BI-1910 agonist, and any combination thereof.

In particular embodiments, the pharmaceutical composition as taught herein further comprises one or more (such as one, two, three or four) immune checkpoint modulators, preferably one or more immune checkpoint inhibitors, selected from the group consisting of ipilimumab, tremelimumab, nivolumab, pembrolizumab, pidilizumab, MEDI0680, atezolizumab, BMS-936559, avelumab (MSB0010718C), durvulamab (MEDI4736), cemiplimab, tislelizumab, sintilimab, JTX-2011, JTX-4014, MEDI-570, GSK3359609, MEDI6469, MEDI6383, MEDI0562, PF-04518660, INCAGN01949, MOXR0916, GSK3174998, TRX518, MEDI1873, GWN323, MK-4166, INCAGN01876, OMP-336B11, PF-05082567, IMP321, LAG525, BMS986016, REGN3767, TSR-033, MGD013, TSR-022, LY3321367, MBG453, OMP-313M32, MTIG7192A/RG6058, JNJ-610588, CA-170, Hu5F9-G4, TTI-621, CC-90002, ALX148, Epacadostat (INCB024360), Pf-06840003, GDC-0919, NLG802, IPH2201 (monalizumab), BMS-986015/IPH-2102 (lirilumab), IPH4102, IPH2101, anti-CD73 antibody from innate pharma with identifier IPH5301, MEDI9447 (oleclumab), CPI-006, TJ004309, BMS-986179, BI-1808, BI-1910 agonist, and any combination thereof.

In particular embodiments, the pharmaceutical composition as taught herein further comprises one or more (such as one, two, three or four) immune checkpoint inhibitors of the group consisting of nivolumab, pembrolizumab, pidilizumab, cemiplimab, tislelizumab, sintilimab, MEDI0680, and any combination thereof, preferably pembrolizumab.

In particular embodiment, the specific ACKR2 modulator and optionally the one or more immune checkpoint inhibitors are the main or only active ingredients of the pharmaceutical composition.

ACKR2, an atypical chemokine receptor, plays a role in a number of inflammatory diseases.

Accordingly, a further aspect provides a specific ACKR2 modulator for use in the treatment of an inflammatory disease or inflammation-associated disease in a subject. The specific ACKR2 modulator is preferably a specific ACKR2 modulator as described herein.

Non-limiting examples of inflammatory diseases or inflammation-associated diseases include arthropathy, inflammatory bowel disease, inflammation-associated lymphangiogenesis, and inflammatory skin disease such as psoriasis.

A similar aspect provides a method of treating a proliferative disease or disorder in a subject comprising administering a therapeutically or prophylactically effective amount of a specific ACKR2 modulator to said subject and the specific ACKR2 modulator for use in the treatment of an inflammatory disease or inflammation-associated disease in a subject.

As described above, present inventors found that exclusively targeting ACKR2 increases the bioavailability of inflammatory chemokines, mainly CCL5 and CXCL10, and allows switching cold immune desert tumors to hot inflamed immune cell-infiltrated tumors eligible for anticancer immunotherapy, such as immune checkpoint modulation therapy, such as immune checkpoint blockade therapy.

A further aspect provides a specific ACKR2 modulator for use in the treatment of a proliferative disease or disorder in a subject. The specific ACKR2 modulator is preferably a specific ACKR2 modulator as described herein.

A similar aspect provides a method of treating a proliferative disease or disorder in a subject comprising administering a therapeutically or prophylactically effective amount of a specific ACKR2 modulator to said subject and the specific ACKR2 modulator for use in the treatment of a proliferative disease or disorder in a subject.

The terms “treat” or “treatment” encompass both the therapeutic treatment of an already developed disease or condition, as well as prophylactic or preventive measures, wherein the aim is to prevent or lessen the chances of incidence of an undesired affliction. Beneficial or desired clinical results may include, without limitation, alleviation of one or more symptoms or one or more biological markers, diminishment of extent of disease, stabilised (i.e., not worsening) state of disease, delay or slowing of disease progression, amelioration or palliation of the disease state, and the like. “Treatment” can also mean prolonging survival as compared to expected survival if not receiving treatment.

The term “proliferative disease or disorder” as used herein generally refers to any disease or disorder characterized neoplastic cell growth and proliferation, whether benign (not invading surrounding normal tissues, not forming metastases), pre-malignant (pre-cancerous), or malignant (invading adjacent tissues and capable of producing metastases). The term proliferative disease generally includes all transformed cells and tissues and all cancerous cells and tissues. Proliferative diseases or disorders include, but are not limited to abnormal cell growth, benign tumors, premalignant or precancerous lesions, malignant tumors, and cancer. Examples of proliferative diseases and/or disorders are benign, pre-malignant, and malignant neoplasms located in any tissue or organ, such as in the prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, or urogenital tract. The term “proliferative disease or disorder” as used herein may be used as a synonym of a neoplastic disease or disorder.

The proliferative disease or disorder (i.e. neoplastic disease or disorder) may be a tumor or may be characterized by the presence of a tumor. As used herein, the terms “tumor” or “tumor tissue” refer to an abnormal mass of tissue that results from excessive cell division. A tumor or tumor tissue comprises “tumor cells” which are neoplastic cells with abnormal growth properties and no useful bodily function. Tumors, tumor tissue and tumor cells may be benign, pre-malignant or malignant, or may represent a lesion without any cancerous potential. A tumor or tumor tissue may also comprise “tumor-associated non-tumor cells”, e.g., vascular cells which form blood vessels to supply the tumor or tumor tissue. Non-tumor cells may be induced to replicate and develop by tumor cells, for example, the induction of angiogenesis in a tumor or tumor tissue.

The proliferative disease or disorder (i.e. neoplastic disease or disorder) may be malignancy. As used herein, the term “malignancy” refers to a non-benign tumor or a cancer.

As used herein, the term “cancer” refers to a malignant neoplasm characterized by deregulated or unregulated cell growth. In certain embodiments, the proliferative disease or disorder may be selected from the group consisting of carcinoma, lymphoma, blastoma, sarcoma, leukemia, and lymphoid malignancies.

In certain further embodiments, the proliferative disease or disorder (i.e. neoplastic disease or disorder) may be selected from the group consisting of skin cancer such as melanoma, colon cancer, rectal cancer, colorectal cancer, bladder cancer, neuroblastoma, squamous cell cancer (e.g., epithelial squamous cell cancer), lung cancer including small-cell lung cancer, non-small cell lung cancer, adenocarcinoma of the lung, squamous carcinoma of the lung and large cell carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, hepatoma, breast cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, head cancer and neck cancer, preferably skin cancer or colorectal cancer, more preferably melanoma or colorectal cancer, even more preferably melanoma (e.g. subcutaneous melanoma).

The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

The proliferative disease or disorder (i.e. neoplastic disease or disorder) may also be a premalignant condition. Premalignant conditions are known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell 1976 (Basic Pathology, 2d Ed., W. B. Saunders Co., Philadelphia, pp. 68-79).

In particular embodiments, the proliferative disease or disorder is melanoma or colorectal cancer, preferably melanoma.

In particular embodiments, the proliferative disease or disorder (i.e. neoplastic disease or disorder) is a primary tumor.

The term “primary tumor” or “original tumor” or “primary lesion” as used herein refers to a tumor at the original site where the tumor first arose.

In particular embodiments, the proliferative disease or disorder (i.e. neoplastic disease or disorder) is not a metastasis (i.e. secondary tumor).

In particular embodiments, the therapy using the specific ACKR2 modulator is a local therapy, for example specifically directed to the tumor or tumor cells. This may be achieved by local administration of the specific ACKR2 modulators, local expression and/or local release of the specific ACKR2 modulator.

In particular embodiments, the method of treating a proliferative disease or disorder in a subject comprising administering a therapeutically or prophylactically effective amount of a specific ACKR2 modulator to said subject is combined with anticancer immunotherapy. Non-limiting examples of anticancer immunotherapy include immune checkpoint modulation therapy (e.g. immune checkpoint blockade therapy, such as a CD3-targeted bispecific antibody, anti-NKG2A-based immunotherapy, anti-KIR based immunotherapy, anti-CD47-based immunotherapy, anti-CD24-based immunotherapy, anti-CD73-based immunotherapy, anti-TNFR2-based immunotherapy, or anti-SIRPα-based immunotherapy), monoclonal antibodies (e.g. alemtuzumab or trastuzumab), chimeric antigen receptor (CAR)-T cell therapy, oncolytic viruses, vaccination with a cancer vaccine, adoptive cell transfer, and stimulator of interferon genes (STING)-based immunotherapy, such as treatment with a STING agonist.

In particular embodiments, the method of treating a proliferative disease or disorder in a subject comprises administering a therapeutically or prophylactically effective amount of a specific ACKR2 modulator to said subject in combination with a therapeutically or prophylactically effective amount of one or more anticancer immunotherapies selected from the group consisting of an immune checkpoint modulator (such as a CD3-targeted bispecific antibody, anti-NKG2A antibody, anti-KIR antibody, anti-CD47 antibody, anti-CD24 antibody, anti-CD73 antibody, anti-TNFR2 antibody, or anti-SIRPα antibody), monoclonal antibodies (e.g. alemtuzumab or trastuzumab), chimeric antigen receptor T cells, an oncolytic virus, a cancer vaccine, a STING agonist and autologous or heterologous tumor-infiltrating lymphocytes to said subject.

In particular embodiments, the method of treating a proliferative disease or disorder in a subject comprises administering a therapeutically or prophylactically effective amount of a specific ACKR2 modulator to said subject in combination with a therapeutically or prophylactically effective amount of one or more of the group consisting of an immune checkpoint inhibitor (such as a CD3-targeted bispecific antibody, anti-NKG2A antibody, anti-KIR antibody, anti-CD47 antibody, anti-CD24 antibody, anti-CD73 antibody, anti-TNFR2 antibody, or anti-SIRPα antibody), monoclonal antibodies (e.g. alemtuzumab or trastuzumab), chimeric antigen receptor T cells, an oncolytic virus, a cancer vaccine, a STING agonist and autologous or heterologous tumor-infiltrating lymphocytes to said subject.

In particular embodiments, the method of treating a proliferative disease or disorder in a subject comprises administering a therapeutically or prophylactically effective amount of a specific ACKR2 modulator to said subject in combination with a therapeutically or prophylactically effective amount of one or more immune checkpoint modulators, preferably one or more immune checkpoint inhibitors.

The term “therapeutically effective amount” as used herein, refers to an amount of therapeutic agent that elicits the biological or medicinal response in a subject that is being sought by a surgeon, researcher, veterinarian, medical doctor or other clinician, which may include inter alia alleviation of the symptoms of the disease or condition being treated. The term “prophylactically effective amount” refers to an amount of the prophylactic agent that inhibits or delays in a subject the onset of a disorder as being sought by a researcher, veterinarian, medical doctor or other clinician. Methods are known in the art for determining therapeutically and/or prophylactically effective amounts of the therapeutic or prophylactic agent as described herein.

A further aspect provides a specific ACKR2 modulator for decreasing tumor growth, for decreasing tumor cell proliferation, for shrinking or decreasing the volume of a tumor (e.g. by at least 5%, at least 10%, at least 20%, at least 30%, at least 40%, at least 50%, at least 60%, at least 70%, at least 80%, at least 90%, or at least 95%, preferably at least 50%) and/or for decreasing the weight of a tumor (e.g. by at least 5%, at least 10%, at least 20%, at least 30%, at least 40%, at least 50%, at least 60%, at least 70%, at least 80%, at least 90%, or at least 95%, preferably at least 50%) in a subject.

The infiltration of cytotoxic immune cells, notably T-lymphocytes, into the tumor bed relies on the establishment of inflamed tumor microenvironment. Indeed, according to the inflammatory status, two major subsets of advanced solid tumors can be identified: tumors with a T cell-inflamed or “hot” microenvironment, which display a signature of a pre-existing adaptive immune response, and non-T cell-inflamed or “cold” microenvironment, which lack evidence of a pre-existing adaptive immune response. Inducing a signature of such adaptive immune response can ultimately lead to reprograming “cold, immune desert” tumor microenvironment to a “hot, immune receptive” one.

Furthermore, cancer growth and progression are associated with immune suppression. Cancer cells have the ability to activate different immune checkpoint pathways that harbor immunosuppressive functions. Inhibiting the cancer cells' ability to suppress the immune system is of interest in the treatment of cancer. Many of the immune checkpoints are initiated by ligand-receptor interactions, and therefore can be readily blocked by antibodies or modulated by recombinant forms of ligands or receptors, also known as immune checkpoint inhibitors.

As described elsewhere herein present inventors found that specific ACKR2 modulators can be used for reprogramming a cold tumor to a hot tumor by increasing the recruitment of NK and CD8+ T cells into the tumor bed. Such increase in NK and CD8+ T cells in the tumor bed can also improve the response of a subject to anticancer immunotherapy, preferably to an immune checkpoint modulator, more preferably an immune checkpoint inhibitor, as a durable clinical response to immune checkpoint modulators, preferably immune checkpoint inhibitors, is dependent on the infiltration of cytotoxic immune cells into the tumor bed.

Accordingly, also disclosed herein is a specific ACKR2 modulator for use in improving the response of a subject to anticancer immunotherapy, preferably to an immune checkpoint modulator, more preferably to an immune checkpoint inhibitor.

The improvement of the response of a subject to anticancer immunotherapy, such as to an immune checkpoint modulator, such as an immune checkpoint inhibitor, may be determined by methods known in the art, such as by the conventional WHO criteria, for example as described in Miller AB, Hoogstraten B, Staquet M, Winkler A. Reporting results of cancer treatment. Cancer. 1981; 47:207-214, Response Evaluation Criteria in Solid Tumors (RECIST), for example as described in Therasse P, et al. New guidelines to evaluate the response to treatment in solid tumors. European Organization for Research and Treatment of Cancer, National Cancer Institute of the United States, National Cancer Institute of Canada. J Natl Cancer Inst. 2000; 92:205-216 or Eisenhauer E A, et al. New response evaluation criteria in solid tumors: revised RECIST guideline (version 1.1) Eur J Cancer. 2009; 45:228-247, by determining the level of biomarkers of immunotherapy response such as the expression level of PD-L1, or by determining the number of tumor-infiltrating lymphocytes, such as CD8+ T cells. More particularly, the improvement of the response of a subject to an immune checkpoint modulator, such as an immune checkpoint inhibitor, may be determined by determining the ability of the immune checkpoint modulator, such as an immune checkpoint inhibitor, to enhance the proliferation, migration, persistence and/or cytoxic activity of CD8+ T cells.

In particular embodiments, the specific ACKR2 modulator improves the response of a subject to anticancer immunotherapy, such as to an immune checkpoint modulator, preferably to an immune checkpoint inhibitor, by at least 1%, at least 5%, at least 10%, at least 25% at least 50%, at least 60%, at least 70%, at least 80%, at least 90%, at least 100%, or greater than 100%, compared to the response of the subject to the anticancer immunotherapy, such as to the immune checkpoint modulator, preferably to the immune checkpoint inhibitor, in absence of the specific ACKR2 modulator.

Anticancer immunotherapy is known in the art and includes immune checkpoint modulation therapy (e.g. immune checkpoint blockade therapy, such as a CD3-targeted bispecific antibody, anti-NKG2A-based immunotherapy, anti-KIR based immunotherapy, anti-CD47-based immunotherapy, anti-CD24 antibody, anti-CD73 antibody, tumor necrosis factor (TNF) receptor 2 (TNFR2) inhibitors (e.g. an anti-TNFR2 antibody), or anti-SIRPα-based immunotherapy), monoclonal antibodies (e.g. alemtuzumab or trastuzumab), chimeric antigen receptor (CAR)-T cell therapy, oncolytic viruses, vaccination with a cancer vaccine, adoptive cell transfer, and stimulator of interferon genes (STING)-based immunotherapy, such as treatment with a STING agonist.

The term “immune checkpoint modulator” as used herein generally refers to any compound inhibiting (e.g. reducing or fully blocking) or stimulating (e.g. increasing) the function of an immune inhibitory checkpoint protein. Non-limiting examples of stimulatory checkpoint modulators include agonists of CD27, OX-40, CD40, ICOS, 4-1BB and GITR.

The term “immune checkpoint inhibitor” as used herein generally refers to any compound inhibiting (e.g. reducing or fully blocking) the function of an immune inhibitory checkpoint protein. Immune checkpoint inhibitors may be peptides, antibodies, nucleic acid molecules and small molecules. As used herein the term “immune checkpoint protein” refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways. Non-limiting examples of inhibitory checkpoint molecules include Adenosine A2A receptor (A2AR), B7-H3, B7-H4, B and T Lymphocyte Attenuator (BTLA), Cytotoxic T-Lymphocyte-Associated protein 4 (CTLA-4), CD277, Indoleamine 2,3-dioxygenase (IDO), immunoglobulin-like receptors (KIRs), Programmed Death ligand 1 (PDL-1), Programmed Death 1 (PD-1), Lymphocyte Activation Gene-3 (LAG-3), T-cell Immunoglobulin domain and Mucin domain 3 (TIM-3) and V-domain Ig suppressor of T cell activation (VISTA). Immune checkpoint inhibitors are known in the art, and are, for example, described in Donini C. et al., Next generation immune-checkpoints for cancer therapy, J Thorac Dis., 2018. 10(S 13): S1581-S 1601. More particularly, non-limiting examples of immune checkpoint inhibitors include agents targeting lymphocyte-activating immune checkpoints such as ICOS, OX40 (CD134), glucocorticoid-induced TNF receptor-related gene (GITR), 4-1BB (CD137), CD40, CD27-CD70; agents targeting lymphocyte-inhibiting immune checkpoints such as LAG3, TIM-3, T cell immunoglobulin and ITIM domain (TIGIT) and VISTA; and agents targeting macrophage and NK immune markers such as CD47, Indoleamine-2,3-dioxygenase (IDO), CD94/NKG2A, and killer immunoglobulin-like receptors (KIRs).

In particular embodiments, the immune checkpoint modulator, preferably the immune checkpoint inhibitor, is selected from the group consisting of CTLA-4 inhibitors, PD-1 inhibitors, PD-L1 inhibitors, CD3 inhibitors, CD24 inhibitors, CD73 inhibitors, ICOS agonists, OX40 agonists, glucocorticoid-induced TNF receptor-related gene (GITR) agonists, CD137 agonists, CD40 agonists, CD27 agonists, CD70 agonists, lymphocyte activation gene 3 (LAG-3) inhibitors, T-cell immunoglobulin- and mucin-domain-containing molecule 3 (TIM-3) inhibitors, T cell immunoglobulin and ITIM domain (TIGIT) inhibitors, V-domain Ig suppressor of T-cell activation (VISTA) inhibitors, CD47 inhibitors, anti-SIRPα inhibitors, Indoleamine-23-dioxygenase (IDO) inhibitors, CD94 inhibitors, NKG2A inhibitors, killer immunoglobulin-like receptor (KIR) inhibitors, CD96 inhibitors, TNFR2 inhibitors, and any combination thereof.

In particular embodiments, the immune checkpoint inhibitor is selected from the group consisting of CTLA-4 inhibitors, PD-1 inhibitors, PD-L1 inhibitors, CD3 inhibitors, CD24 inhibitors, CD73 inhibitors, lymphocyte activation gene 3 (LAG-3) inhibitors, T-cell immunoglobulin- and mucin-domain-containing molecule 3 (TIM-3) inhibitors, T cell immunoglobulin and ITIM domain (TIGIT) inhibitors, V-domain Ig suppressor of T-cell activation (VISTA) inhibitors, CD47 inhibitors, anti-SIRPα inhibitors, Indoleamine-2,3-dioxygenase (IDO) inhibitors, CD94 inhibitors, NKG2A inhibitors, killer immunoglobulin-like receptor (KIR) inhibitors, CD96 inhibitors, TNFR2 inhibitor, and any combination thereof.

In particular embodiments, the immune checkpoint modulator, preferably the immune checkpoint inhibitor, is selected from the group consisting of anti-CTLA-4 antibody, anti-PD-1 antibody, anti-PD-L1 antibody, anti-CD3 antibody (e.g. a CD3-targeted bispecific antibody), anti-CD24 antibody, anti-CD73 antibody, anti-ICOS antibody, anti-OX40 antibody, anti-GITR antibody, anti-CD137 antibody, anti-CD40 antibody, anti-CD27 antibody, anti-CD70 antibody, anti-LAG-3 antibody, anti-TIM-3 antibody, anti-TIGIT antibody, anti-VISTA antibody, anti-CD47 antibody, anti-SIRPα antibody, anti-IDO antibody, anti-CD94 antibody, anti-NKG2A antibody, anti-KIR antibody, anti-CD96 antibody, anti-TNFR2 antibody, and any combination thereof.

In particular embodiments, the immune checkpoint modulator, preferably the immune checkpoint inhibitor, is selected from the group consisting of inhibitory anti-CTLA-4 antibody, inhibitory anti-PD-1 antibody, inhibitory anti-PD-L1 antibody, inhibitory anti-CD3 antibody (e.g. a CD3-targeted bispecific antibody), inhibitory anti-CD24 antibody, inhibitory anti-CD73 antibody, stimulatory anti-ICOS antibody, stimulatory anti-OX40 antibody, stimulatory anti-GITR antibody, stimulatory anti-CD137 antibody, stimulatory anti-CD40 antibody, stimulatory anti-CD27 antibody, stimulatory anti-CD70 antibody, inhibitory anti-LAG-3 antibody, inhibitory anti-TIM-3 antibody, inhibitory anti-TIGIT antibody, inhibitory anti-VISTA antibody, inhibitory anti-CD47 antibody, inhibitory anti-SIRPα antibody, inhibitory anti-IDO antibody, inhibitory anti-CD94 antibody, inhibitory anti-NKG2A antibody, inhibitory anti-KIR antibody, inhibitory anti-CD96 antibody, inhibitory anti-TNFR2 antibody, and any combination thereof.

In particular embodiments, the immune checkpoint inhibitor is selected from the group consisting of inhibitory anti-CTLA-4 antibodies, inhibitory anti-PD-1 antibodies, inhibitory anti-PD-L1 antibodies, inhibitory anti-CD3 antibodies, inhibitory anti-CD24 antibodies, inhibitory anti-CD73 antibodies, inhibitory anti-LAG-3 antibodies, inhibitory anti-TIM-3 antibodies, inhibitory anti-TIGIT antibodies, inhibitory anti-VISTA antibodies, inhibitory anti-CD47 antibodies, inhibitory anti-SIRPα antibodies, inhibitory anti-IDO antibodies, inhibitory anti-CD94 antibodies, inhibitory anti-NKG2A antibodies, inhibitory anti-KIR antibodies, inhibitory anti-CD96 antibodies, TNFR2 antibodies, and any combination thereof.

In particular embodiments, the immune checkpoint modulator, preferably the immune checkpoint inhibitor, is selected from the group consisting of ipilimumab, tremelimumab, nivolumab, pembrolizumab, pidilizumab, MEDI0680, atezolizumab, BMS-936559, avelumab (MSB0010718C), durvulamab (MEDI4736), Cemiplimab, Tislelizumab, Sintilimab, JTX-2011, JTX-4014, MEDI-570, GSK3359609, MEDI6469, MEDI6383, MEDI0562, PF-04518660, INCAGN01949, MOXR0916, GSK3174998, TRX518, MEDI1873, GWN323, MK-4166, INCAGN01876, OMP-336B11, PF-05082567, CP870893, R07009789, MOXR0916, CDX-1140, Varlilumab, SGN-CD70A, IMP321, LAG525, BMS986016, REGN3767, TSR-033, MGD013, TSR-022, LY3321367, MBG453, OMP-313M32, MTIG7192A/RG6058, JNJ-610588, CA-170, Hu5F9-G4, TTI-621, CC-90002, ALX148, Epacadostat (INCB024360), Pf-06840003, GDC-0919, NLG802, IPH2201 (monalizumab), BMS-986015/IPH-2102 (lirilumab), IPH4102, IPH2101, anti-CD73 antibody from innate pharma with identifier IPH5301, MEDI9447 (oleclumab), CPI-006, TJ004309, BMS-986179, BI-1808, BI-1910 agonist, and any combination thereof, preferably selected from the group consisting of ipilimumab, tremelimumab, nivolumab, pembrolizumab, pidilizumab, MEDI0680, atezolizumab, BMS-936559, avelumab (MSB0010718C), durvulamab (MEDI4736), Cemiplimab, Tislelizumab, Sintilimab, JTX-2011, JTX-4014, MEDI-570, GSK3359609, MEDI6469, MEDI6383, MEDI0562, PF-04518660, INCAGN01949, MOXR0916, GSK3174998, TRX518, MEDI1873, GWN323, MK-4166, INCAGN01876, OMP-336B11, PF-05082567, IMP321, LAG525, BMS986016, REGN3767, TSR-033, MGD013, TSR-022, LY3321367, MBG453, OMP-313M32, MTIG7192A/RG6058, JNJ-610588, CA-170, Hu5F9-G4, TTI-621, CC-90002, ALX148, Epacadostat (INCB024360), Pf-06840003, GDC-0919, NLG802, IPH2201 (monalizumab), BMS-986015/IPH-2102 (lirilumab), IPH4102, IPH2101, anti-CD73 antibody from innate pharma with identifier IPH5301, MEDI9447 (oleclumab), CPI-006, TJ004309, BMS-986179, BI-1808, BI-1910 agonist, and any combination thereof.

In particular embodiments, the immune checkpoint inhibitor is selected from the group consisting of ipilimumab, tremelimumab, nivolumab, pembrolizumab, pidilizumab, MEDI0680, atezolizumab, BMS-936559, avelumab (MSB0010718C), durvulamab (MEDI4736), Cemiplimab, Tislelizumab, Sintilimab, JTX-4014, IMP321, LAG525, BMS986016, REGN3767, TSR-033, MGD013, TSR-022, LY3321367, MBG453, OMP-313M32, MTIG7192A/RG6058, JNJ-610588, CA-170, Hu5F9-G4, TTI-621, CC-90002, ALX148, Epacadostat (INCB024360), Pf-06840003, GDC-0919, NLG802, IPH2201 (monalizumab), BMS-986015/IPH-2102 (lirilumab), IPH4102, IPH2101, IPH2101, anti-CD73 antibody from innate pharma with identifier IPH5301, MEDI9447 (oleclumab), CPI-006, TJ004309, BMS-986179, BI-1808, BI-1910 agonist, and any combination thereof.

In particular embodiments, the immune checkpoint modulator, preferably the immune checkpoint inhibitor, is selected from the group consisting of a PD-L1 immune checkpoint inhibitor, a PD-1 immune checkpoint inhibitor, a CTLA-4 immune checkpoint inhibitor, a CD3 inhibitor (e.g. a CD3-targeted bispecific antibody), a NKG2A inhibitor (e.g. an anti-NKG2A antibody), a KIR inhibitor (e.g. an anti-KIR antibody), a CD47 inhibitor (e.g. an anti-CD47 antibody), a CD24 inhibitor (e.g. an anti-CD24 antibody), a CD73 inhibitor (e.g. an anti-CD73 antibody), a TNFR2 inhibitor (e.g. an anti-TFNR2 antibody), and a SIRPα inhibitor (e.g. an anti-SIRPα antibody). Preferably, the immune checkpoint inhibitor is selected from the group consisting of a PD-L1 immune checkpoint inhibitor, a PD-1 immune checkpoint inhibitor or a CTLA-4 immune checkpoint inhibitor, more preferably a PD-1 immune checkpoint inhibitor. Non-limiting examples of CTLA-4 immune checkpoint inhibitors include anti-CTLA-4 antibodies such as ipilimumab and tremelimumab, anti-CD28 antibodies, anti-CTLA-4 adnectins and combinations thereof. Non-limiting examples of PD-1 immune checkpoint inhibitors include anti-PD-1 antibodies such as nivolumab, pembrolizumab, pidilizumab, cemiplimab, tislelizumab, sintilimab and MEDI0680. Non-limiting examples of PD-L1 immune checkpoint inhibitors include anti-PDL-1 antibodies such as atezolizumab, BMS-936559, Durvalumab (MEDI4736), avelumab (MSB0010718C), and combinations thereof.

In particular embodiments, the immune checkpoint modulator, preferably the immune checkpoint inhibitor, is selected from the group consisting of an anti-CTLA-4 antibody, an anti-CD28 antibody, an anti-PD-1 antibody, an anti PDL-1 antibody, a CD3-targeted bispecific antibody, an anti-NKG2A antibody, an anti-KIR antibody, an anti-CD47 antibody, an anti-CD24 antibody, an anti-CD73 antibody, an anti-TFNR2 antibody and an anti-SIRPα antibody, and combinations thereof. Preferably, the immune checkpoint modulator, preferably the immune checkpoint inhibitor, is selected from the group consisting of nivolumab, pembrolizumab, pidilizumab, cemiplimab, tislelizumab, sintilimab and MEDI0680, preferably pembrolizumab, ipilimumab, tremelimumab, atezolizumab, BMS-936559, durvulamab (MEDI4736), avelumab (MSB0010718C), and combinations thereof, more preferably pembrolizumab.

The term “subject” or “patient” as used herein typically and preferably denotes humans, but may also encompass reference to non-human animals, preferably warm-blooded animals, more preferably vertebrates, even more preferably mammals, such as, e.g., non-human primates, rodents, canines, felines, equines, ovines, porcines, and the like. Particularly intended are subjects known or suspected to have a proliferative disease or disorder. Suitable subjects may include ones presenting to a physician for a screening for a proliferative disease or disorder and/or with symptoms and signs indicative of a proliferative disease or disorder.

In particular embodiments, the subject is diagnosed with or assumed to have a proliferative disease or disorder, preferably cancer.

In particular embodiments, the subject is diagnosed with or assumed to have a proliferative disease or disorder characterized by a low or non-existing tumor infiltration of NK cells, CD8+ T cells, CD4+ cells and/or CD4 effector cells, preferably characterized by a low or non-existing tumor infiltration of NK cells and/or CD8+ T cells.

In particular embodiments, the subject is diagnosed with or assumed to have melanoma characterized by a ratio of the number of CD8+ cells over the number of T regulator cells of less than 0.2, preferably less than 0.1.

In particular embodiments, the subject is diagnosed with or assumed to have a proliferative disease or disorder not responding to anticancer immunotherapy, preferably not responding to immune checkpoint modulation therapy, more preferably not responding to immune checkpoint blockade therapy, such as not responding to an immune checkpoint inhibitor.

As used herein the term “non-responder” in the context of the present disclosure refers to a subject that will not achieve a response, i.e. a subject where the proliferative disease or disorder is not eradicated, reduced or improved upon administration of the anticancer immunotherapy, such as immune checkpoint modulation therapy, such as immune checkpoint blockade therapy. The term “non-responder” may also include patients having a stabilized proliferative disease or disorder.

In particular embodiments, the subject is diagnosed with or assumed to have melanoma or colorectal cancer.

In particular embodiments, the subject is diagnosed with or assumed to have melanoma or colorectal cancer non-responsive to a PD-1 inhibitor, preferably pembrolizumab.

In particular embodiments, the method of treating a proliferative disease in a subject as taught herein comprises the steps of:

a) quantifying the density of NK and/or CD8+ T cells in a tumor tissue sample obtained from the subject;

b) comparing the density quantified at step a) with a predetermined reference value; and

c) administering to the subject a therapeutically effective amount of a specific ACKR2 modulator and one or more immune checkpoint modulators, preferably one or more immune checkpoint inhibitors, when the density quantified at step a) is lower than the predetermined reference value.

Tumor infiltration of NK and/or CD 8+ T cells or the density of NK and/or CD8+ T cells in a tumor tissue sample can be determined by any method known in the art. For example, by immunohistochemistry (IHC) comprising contacting the tumor tissue sample with a binding partner (e.g. an antibody) specific for a cell surface marker of NK and/or CD 8+ T cells.

The terms “sample” or “biological sample” as used herein include any biological specimen obtained and isolated from a subject. Samples may include, without limitation, organ tissue (i.e., tumor tissue), whole blood, plasma, serum, whole blood cells, red blood cells, white blood cells (e.g., peripheral blood mononuclear cells), saliva, urine, stool (i.e., faeces), tears, sweat, sebum, nipple aspirate, ductal lavage, tumor exudates, synovial fluid, cerebrospinal fluid, lymph, fine needle aspirate, amniotic fluid, any other bodily fluid, cell lysates, cellular secretion products, inflammation fluid, semen and vaginal secretions. Preferably, a sample may be readily obtainable by minimally invasive methods, such as blood collection or tissue biopsy, allowing the removal/isolation/provision of the sample from the subject. The term “tissue” as used herein encompasses all types of cells of the human body including cells of organs but also including blood and other body fluids recited above. The sample may be a tumor tissue sample. The term “tumor tissue sample” means any tissue tumor sample derived from the patient. The tumor sample may result from the tumor resected from the patient or from a biopsy performed in the primary tumor of the patient. The sample can be fresh, frozen, fixed (e.g., formalin fixed), or embedded (e.g., paraffin embedded).

A further aspect provides an in vitro method for identifying an agent useful as a therapeutic, such as useful for the treatment of a proliferative disease or disorder in a subject, said method comprising determining whether a test agent (e.g. a small compound) modulates the biological activity of ACKR2.

In particular embodiments, said method comprises a step of determining whether a test agent increases the biological activity of ACKR2. In other words, said method comprises a step of determining whether a test agent is an ACKR2 agonist.

In particular embodiments, said method comprises
contacting the test agent with a cell capable inducing β-arrestin 1 and/or β-arrestin 2 recruitment to ACKR2 and measuring β-arrestin 1 and/or β-arrestin 2 recruitment to ACKR2, and
determining that the agent is useful as a therapeutic when the agent increases β-arrestin1 and/or β-arrestin 2 recruitment to ACKR2.

The interaction between ACKR2 and β-arrestin-1 and/or β-arrestin-2 can be determined by any established analytical technique for determining protein-protein binding, such as co-immunoprecipitation, bimolecular fluorescence complementation, label transfer, tandem affinity purification, chemical cross-linking, fluorescence resonance energy transfer and nanoluciferase complementation assays (e.g. NanoBiT, Promega or NanoBRET, Promega) as described elsewhere herein. In particular embodiments, said method comprises a step of determining whether a test agent decreases or eliminates the biological activity of ACKR2. In particular embodiments, said method comprises a step of determining whether a test agent is an ACKR2 antagonist.

The antagonist activity of a test agent can be determined using β-arrestin recruitment assays in the presence of a known agonist ligand at concentrations equivalent to EC₅₀ or EC₈₀ or can be determined by its ability to compete with the binding of a labeled ACKR2 ligand (e.g. a fluorescent or radiolabeled ACKR2 ligands).

In particular embodiments, said method comprises
contacting the test agent with a cell capable inducing β-arrestin 1 and/or β-arrestin 2 recruitment to ACKR2 in the presence of an ACKR2 ligand, preferably an ACKR2 agonist ligand, and measuring (3-arrestin 1 and/or β-arrestin 2 recruitment to ACKR2, and
determining that the agent is useful as a therapeutic when the agent reduces or eliminates β-arrestin1 and/or β-arrestin 2 recruitment to ACKR2 in the presence of the ACKR2 ligand, preferably an ACKR2 agonist ligand.

In particular embodiments, the ACKR2 ligand, preferably an ACKR2 agonist ligand, is present at a concentration equivalent to EC₅₀ or EC₈₀.

In particular embodiments, said method comprises
contacting the test agent with a cell capable inducing β-arrestin 1 and/or β-arrestin 2 recruitment to ACKR2 in the presence of CCL2, CCL3, CCL4, CCL5, CCL7, CCL8, CCL11, CCL13, CCL14, CCL17, CCL22 and/or CXCL10 and measuring β-arrestin 1 and/or β-arrestin 2 recruitment to ACKR2, and
determining that the agent is useful as a therapeutic when the agent reduces or eliminates β-arrestin1 and/or β-arrestin 2 recruitment to ACKR2 in the presence of CCL2, CCL3, CCL4, CCL5, CCL7, CCL8, CCL11, CCL13, CCL14, CCL17, CCL22 and/or CXCL10.

The interaction between ACKR2 and β-arrestin-1 and/or β-arrestin-2 can be determined by any established analytical technique for determining protein-protein binding, such as co-immunoprecipitation, bimolecular fluorescence complementation, label transfer, tandem affinity purification, chemical cross-linking, fluorescence resonance energy transfer and nanoluciferase complementation assays (e.g. NanoBiT, Promega or NanoBRET, Promega) as described elsewhere herein.

In particular embodiments, said method further comprises determining whether the test agent specifically binds to ACKR2, and optionally determining whether the test agent is capable of binding to any other chemokine receptor than ACKR2, such as a chemokine receptor selected from the group consisting of CCR1 (e.g. with UniProt accession number P32246), CCR2 (e.g. with UniProt accession number P41597) such as CCR type 2A (CCR2A) or CCR type 2B (CCR2B), CCR3 (e.g. with UniProt accession number P51677), CCR4 (e.g. with UniProt accession number P51679), CCR5 (e.g. with UniProt accession number P51681), CCR6 (e.g. with UniProt accession number P51684), CCR7 (e.g. with UniProt accession number P32248), CCR8 (e.g. with UniProt accession number P51685), CCR9 (e.g. with UniProt accession number P51686), CCR10 (e.g. with UniProt accession number P46092), CXCR1 (e.g. with UniProt accession number P25024), CXCR2 (e.g. with UniProt accession number P25025), CXCR3 (e.g. with UniProt accession number P49682) such as CXCR type 3A (CXCR3A) and CXCR type 3B (CXCR3B), CXCR4 (e.g. with UniProt accession number P61073), CXCR5 (e.g. with UniProt accession number P32302), CXCR6 (e.g. with UniProt accession number 000574), CXCR8 (e.g. with UniProt accession number Q9HC97), XCR1 (e.g. with UniProt accession number P46094), CX3CR1 (e.g. with UniProt accession number P49238), ACKR1 (e.g. with UniProt accession number Q16570), ACKR3 (e.g. with UniProt accession number P25106) and ACKR4 (e.g. with UniProt accession number Q9NPB9).

In particular embodiments, said method comprises determining whether the test agent specifically binds to ACKR2 in the presence of a ACKR2 ligand, such as in the presence of CCL2, CCL3, CCL4, CCL5, CCL7, CCL8, CCL11, CCL13, CCL14, CCL17, CCL22 and/or CXCL10.

In particular embodiments, the ACKR2 ligand is labelled, such as fluorescently labelled or radiolabeled.

In particular embodiments, said method further comprises determining whether the test agent inhibits, reduces and/or prevents the interaction between ACKR2 and CCL5 and/or CXCL10.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The herein disclosed aspects and embodiments of the invention are further supported by the following non-limiting examples.

### EXAMPLES

### Example 1. Materials and Methods for Examples 2 to 10

Chemokines and Antibodies

All the unlabelled chemokines were purchased from PeproTech. hACKR2-specific mAb (clone 196124 (R&D Systems). CXCL10 was labelled with Cy5 using the Amersham QuickStain Protein Labeling Kit (GE Healthcare Life Sciences).

Chemokine Processing by Dipeptidyl Peptidase 4

CCL5, CCL2, CXCL10, CXCL11 and CXCL12 chemokines (9 μM) were incubated with recombinant dipeptidyl peptidase 4 (CD26) (200 U) in Tris/HCl 50 mM pH7.5+1 mM EDTA for 2 hours at 37° C.

Cells and Reagents

The B16-F10 cell line was purchased from ATCC. DMEM, FBS, and antibiotics were obtained from Life Technologies. Cell lines were cultured in DMEM supplemented with 10% Fetal Bovine Serum (FBS) and 1% Penicillin/Streptomycin at 37° C. and 5% CO2. The cell lines were mycoplasma-free based on tests with a Mycoalert kit (Lonza). The B16-F10 cells were transfected according to the manufacturer's protocol with either control shRNA (sc-108080) or Ackr2 (sc-60338-V) Lentiviral Particles purchased from Santa Cruz Biotechnology. HEK293T cells were from ATCC and NIH AIDS Reagent Program. HEK cell line stably expressing hACKR2 was established by XtremeGene9 transfection (Roch) of HEK293T cells with pIRES-puro vector (Addgene) encoding human ACKR2, subsequent puromycin selection (5 μg/mL). Receptor surface expression was verified by flow cytometry using hACKR2-specific mAb (clone 196124). Cells were grown in Dulbecco's modified Eagle medium supplemented with 15% fetal bovine serum, penicillin (100 IU/mL) and streptomycin (100 μg/mL). The absence of CXCR3 at the cell surface was confirmed using mAb clone 106 and the corresponding isotype control (Biologend).

β-Arrestin Recruitment Assays

Chemokine-induced β-arrestin recruitment to ACKR2 and CXCR3 was monitored by NanoLuc complementation assay (NanoBiT, Promega; Dixon, A. S., et al., NanoLuc Complementation Reporter Optimized for Accurate Measurement of Protein Interactions in Cells. ACS Chem Biol, 2016. 11(2): p. 400-8) as previously described in Szpakowska, M., et al., Mutational analysis of the extracellular disulphide bridges of the atypical chemokine receptor ACKR3/CXCR7 uncovers multiple binding and activation modes for its chemokine and endogenous non-chemokine agonists. Biochem Pharmacol, 2018. 153: p. 299-309 and Szpakowska, M., et al., Different contributions of chemokine N-terminal features attest to a different ligand binding mode and a bias towards activation of ACKR3/CXCR7 compared with CXCR4 and CXCR3. Br J Pharmacol, 2018. 175(9): p. 1419-1438. In brief, 5×10⁶ HEK293T cells were plated in 10 cm-culture dishes and 24 hours later co-transfected with pNBe vectors encoding ACKR2 or CXCR3 C-terminally tagged to SmBiT and human β-arrestin-1 N-terminally fused to LgBiT. 24 hours post-transfection cells were harvested, incubated 25 minutes at 37° C. with 200-fold diluted Nano-Glo Live Cell substrate and distributed into white 96-well plates (5×10⁴ cells per well). Upon addition of chemokines at the indicated concentrations, β-arrestin recruitment to ACKR2 and CXCR3 was evaluated with a Mithras LB940 luminometer (Berthold Technologies). Each point corresponds to averaged values acquired for 20 min and represented as fold increase over untreated cells.

Chemokine-induced β-arrestin recruitment to ACKR2 was also monitored by NanoBRET using mNeonGreen as acceptor molecule. HEK293T cells were co-transfected with pNeonGreen and pNLF vectors, encoding ACKR2 C-terminally fused to mNeonGreen and β-arrestin-1 N-terminally fused to Nanoluciferase. Twenty-four hours post-transfection cells were harvested and upon simultaneous addition of Nano-Glo Live Cell substrate (1:200) and chemokines, BRET signal was measured with a Mithras LB940 luminometer (Berthold Technologies) using a 460/70 BP filter for Nanoluciferase and a 515/40 BP filter for mNeonGreen signal.

Chemokine Binding

HEK293T and HEK-ACKR2 cells were incubated with CXCL10-Cy5 at indicated concentrations for 45 minutes at 37° C., then washed twice with FACS buffer (PBS, 1% BSA, 0.1% NaN₃). Dead cells were excluded using Zombie Green viability dye (BioLegend). ACKR2-negative HEK293T cells were used to evaluate non-specific binding of CXCL10-Cy5. For binding competition with unlabelled chemokines (50 nM), the signal obtained for CXCL10-Cy5 in the absence of unlabelled chemokines was used to define 100% binding. Ligand binding was quantified by mean fluorescence intensity on a BD FACS Fortessa cytometer (BD Biosciences).

Chemokine Scavenging Assay

Chemokine depletion from the extracellular space was quantified by ELISA. HEK293T and HEK.hACKR2 cells were incubated 8 hours at 37° C. with chemokines at concentrations ranging from 0.03 nM to 30 nM. Chemokine scavenging by ACKR2 was evaluated by quantifying the concentration of chemokines (e.g. CXCL10, CCL5 and CXCL11) remaining in the supernatant using commercially available ELISA kits (R&D Systems, Biolegend, Peprotech). Chemokine scavenging was expressed as the percentage of chemokine compared to untreated cells.

Chemokine Internalization

Chemokine internalization was visualized by imaging flow cytometry using labelled CXCL10 as previously described (Meyrath, M. et al. The atypical chemokine receptor ACKR3/CXCR7 is a broad-spectrum scavenger for opioid peptides. Nat Commun 11, 3033, (2020)). HEK or HEK.ACKR2 cells were incubated 15 minutes at 37° C. in the presence or absence of non-labelled chemokines (200 nM) after which Cy5-labeled CXCL10 was added (100 nM) for 40 minutes at 37° C. Cells were washed twice with FACS buffer. Dead cells were excluded using Zombie Green viability dye (1:3000, BioLegend). Images of 1×10⁴ in-focus living single cells were acquired with an ImageStream MKII imaging flow cytometer (Amnis) using 60× magnification. Samples were analyzed using Ideas6.2 software. The number of spots per cell was determined using a mask-based software wizard.

Inhibition of Chemokine Scavenging by Anti-mACKR2

HEK.mACKR2 cells were incubated 8 hours at 37° C. with chemokines (3, 10 or 30 nM) in the presence or absence of the polyclonal anti-mACKR2 antibody (20 μg/ml) (Abcam, ab1656) or the goat IgG control antibody (ab37373). Chemokine scavenging by ACKR2 was evaluated by quantifying the concentration of mCXCL10 and mCCL5 remaining in the supernatant using commercially available ELISA kits (R&D Systems). Inhibition of mCXCL10 and mCCL5 scavenging by anti-mACKR2 was expressed as the percentage relative to cells incubated in the absence of the antibody.

In Vivo Study Approval

Mice were treated in accordance with European Union guidelines, and the in vivo experimentation protocols were approved by the LIH ethical committee, Animal Welfare Society Luxembourg (agreements n. LECR-2017-02; and LECR-2018-12).

In Vivo Tumor Growth in Mice

C57BL/6 mice (7 weeks old) and NSG mice were purchased from Janvier and housed in pathogen-free conditions for one week prior to the experiments. Mice were injected subcutaneously in the right flank with B16-F10 cells diluted in 100 μl of PBS. Tumor volume was measured using a caliper every other day and estimated as follows: Volume (cm3)=(width) 2×length×0.5. Mice that did not develop tumors or developed tumors larger than the threshold defined in the in vivo experimentation protocols approved by the animal welfare committee of Luxembourg Institute of Health (volume≥2000 mm3) were excluded.

Tumor Immunophenotyping and Flow Cytometry Analysis

Tumors were dissected and mechanically dissociated into small, <4-mm fragments with a scalpel, followed by digestion with mouse tumor dissociation kit (Miltenyi Biotec) for 45 min at 37° C. After single-cell suspensions were obtained, red blood cells were removed by ACK (10-548E, Lonza). The cells were next counted using a Countess Automated Cell Counter (Invitrogen) and blocked for 30 minutes on ice with Fc block (TruStain fcX™ (anti-mouse CD16/32) Antibody 101320 Biolegend). The samples were first stained for surface markers for lymphoid immune populations followed by intracellular staining. For FoxP3 and intracellular staining, True-Nuclear™ Transcription Factor Buffer Set 424401 Biolegend was used according to the manufacturer's recommended protocol. The following antibodies were purchased from Biolegend: FITC anti-mouse CD45, Brilliant Violet 785 anti-mouse CD3, APC anti-mouse CD8a, APC/Fire 750 anti-mouse CD4, PE/Cy7 anti-mouse CD49b (pan-NK cells), PE/Cy7 anti-mouse NK-1.1 Antibody, Brilliant Violet 605 anti-mouse CD69, PE/Cy5 anti-mouse CD25, Brilliant Violet 421 anti-mouse FOXP3, and PE/Dazzle 594 anti-mouse CD279 (PD-1). LIVE/DEAD Fixable Blue Dead Cell Stain Kit (ThermoFisher Scientific) was used as a viability dye. Single stains were performed for compensation controls, FMO controls to check for fluorescence spread and isotype controls were used to determine the level of non-specific binding.

In Vivo Blocking Antibodies

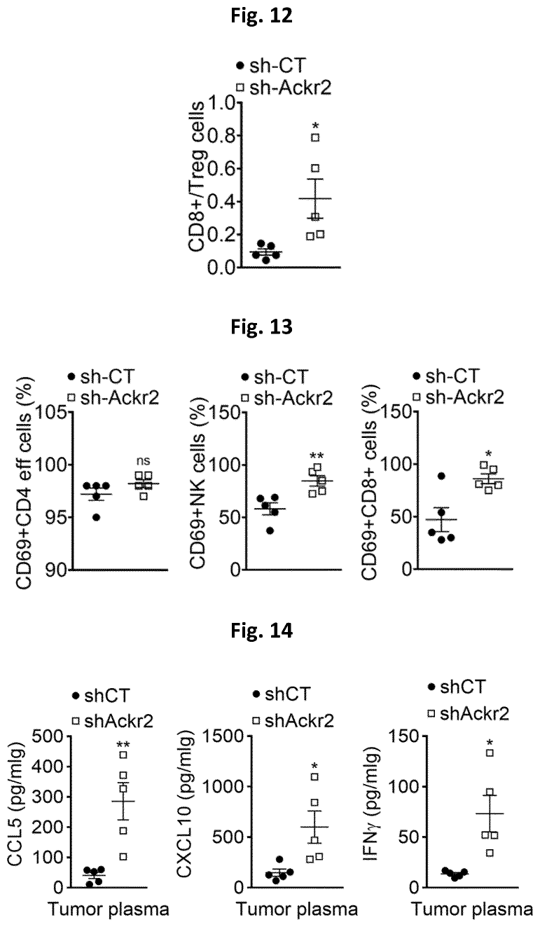

The InVivoMab anti-mouse PD-1 (CD279) (BE0273) and InVivoMab rat IgG2a isotype control (BE0089) were purchased from BioXCell, diluted in InVivoPure pH 7.0 Dilution Buffer (IP0070) and administered as indicated in the FIG. 12.

Flow Cytometry Analysis for Ackr2 on B16-F10 Cells.

Flow cytometry was performed using anti-Akcr2 antibody (Abcam) and analysed on CytoFLEX (Beckman Coulter). Data were further analyzed by Flow Jo 7.6.5 software (Tree Star).

RNA Extraction and SYBR Green Real-Time (RT)-qPCR.

Total RNAs were extracted using the miRCURY RNA isolation kit (300110; Exiqon). RNAs were quantified by Nanodrop. RNA (1 μg) from each sample was reverse-transcribed using a reverse-transcription reaction mix (Eurogentec). The reverse transcription was performed at 48° C. for 30 min. The resulting cDNA was subjected to amplification by qPCR using power SYBR green PCR master mix (Life Technologies). The RPL13 gene encoding Ribosomal Protein L13 was used as internal control. For mouse Ackr2, primers were purchased from Qiagen.

ELISA

For tumor plasma preparation, tumors were dissociated in DMEM medium, and then centrifuged to collect the supernatant. The supernatant was concentrated with Protein Concentrator PES, 3K MWCO (88526, Fisher Scientific) according to manufacturer's protocol. For in vitro cell supernatants, B16-F10 cells were plated in 6-well dishes, CCL5, CXCL10 and IFNγ were quantified using mouse CCL5/RANTES DuoSet ELISA (DY478-05; R&D Systems), mouse CXCL10/IP-10/CRG-2 DuoSet ELISA (DY466-05; R&D Systems), and Mouse IFN-gamma DuoSet ELISA (DY485-05; R&D Systems).

Data and Statistical Analysis

Statistical analyses were conducted using GraphPad Prism software (version 7.00 or 8.0.1). The error bars represent the standard error of the mean (SEM). The data are represented as the average±SEM. p values were calculated using an unpaired, two-tailed Student's t-test to compare the two groups. A p-value less than 0.05 was considered statistically significant. * p<0.05; ** p<0.01; *** p<0.001; **** p<0.0001, ns: not significant. Concentration-response curves were fitted to the four-parameter Hill equation using an iterative, least-squares method (GraphPad Prism version 8.0.1) to provide pEC₅₀, pIC₅₀, EC₅₀ or IC₅₀ values and standard errors of the mean. All curves were fitted to data points generated from the mean of at least three independent experiments.

### Example 2. ACKR2 is a Scavenger for CXCL10

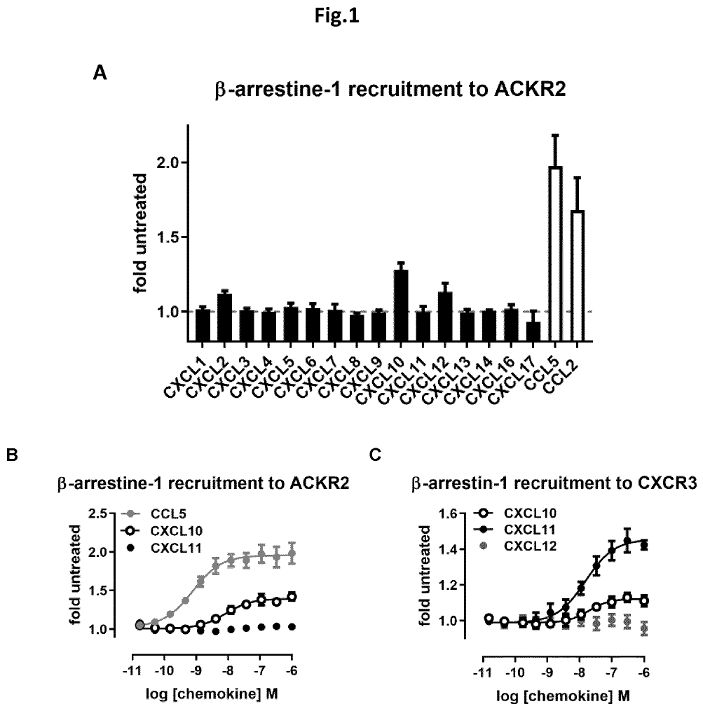

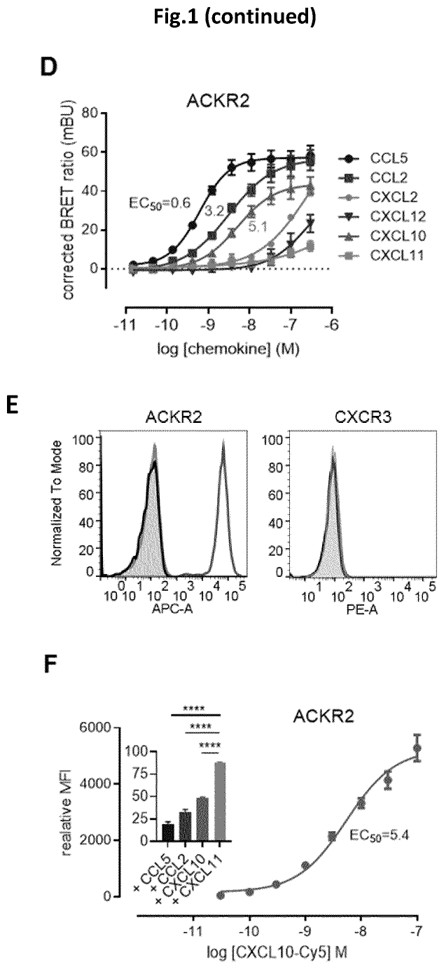

The ability of human CXC chemokines CXCL1, CXCL2, CXCL3, CXCL4, CXCL5, CXCL6, CXCL7, CXCL8, CXCL9, CXCL10, CXCL11, CXCL12, CXCL13, CXCL14, CXCL16, CXCL17, CCL5 and CCL2 to active ACKR2 was assessed in HEK293T cells using the ultra-sensitive Nanoluciferase complementation-based assay (NanoBiT) based on β-arrestin recruitment. CCL5 and CCL2 that act as full and partial agonists on ACKR2 were used as positive controls. Present inventors' screening revealed that at least three CXC chemokines, namely CXCL2, CXCL10 and CXCL12 are capable of inducing β-arrestin-1 recruitment to ACKR2 with CXCL10 giving the highest signal (FIG. 1A).

To characterise further the interactions of these CXC chemokines with ACKR2 and to establish whether their activity towards ACKR2 may be of physiological relevance, especially in light of a possible scavenging function, we performed pharmacological analysis, investigating the potency and efficacy of the chemokine hits towards ACKR2 and CXCR3, the only receptor reported for CXCL10. Present inventors observed that, just like for its signaling receptor CXCR3, CXCL10 act as a partial agonist of ACKR2 inducing about 30% of the maximal response observed with the full agonist CCL5 and CXCL11, respectively (FIG. 1 B-C). The potency of CXCL10 towards ACKR2 (EC₅₀=8.2 nM, pEC₅₀=8.08±0.14) appears approximately 3 times stranger than towards CXCR3 (EC₅₀=24.9 nM, pEC₅₀=7.60±0.12), consistent with a potential scavenging role of ACKR2. In NanoBRET, the potency of CXCL10 (EC₅₀=5.1 nM, pEC₅₀=8.29±0.11) was close to that of CCL2 and its efficacy reached approximately 70% of the maximal signal monitored with CCL5 (FIG. 1D). Fluorescently labelled CXCL10 also strongly and specifically bound to HEK293T cells expressing ACKR2 but not its classical receptor CXCR3 (FIG. 1E-F) and was only displaced by ACKR2-related chemokines CCL5, CCL2 and by CXCL10 itself (FIG. 1F inset). Moreover, the screening of CXCL10 on all the 23 chemokine receptors showed that CXCR3 and ACKR2 are the only two receptors activated by CXCL10 (FIG. 2A-B).

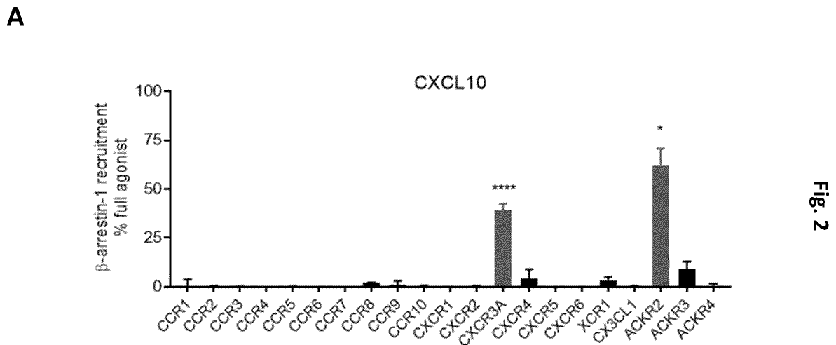

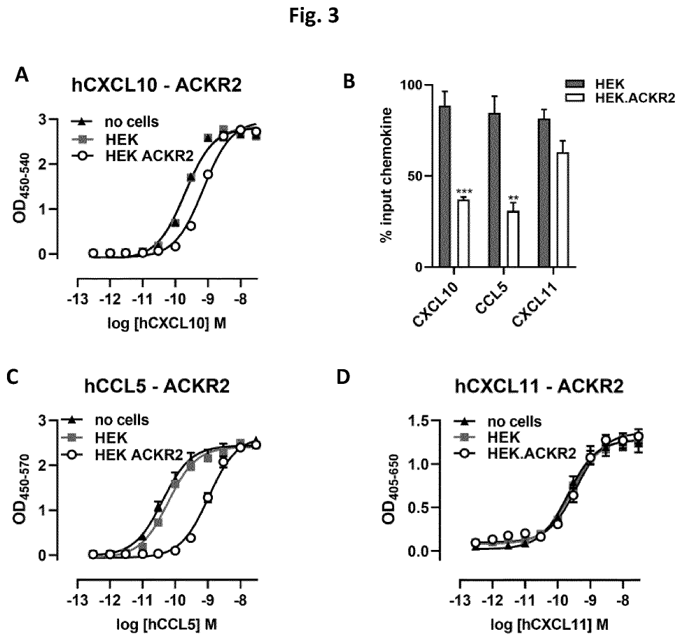

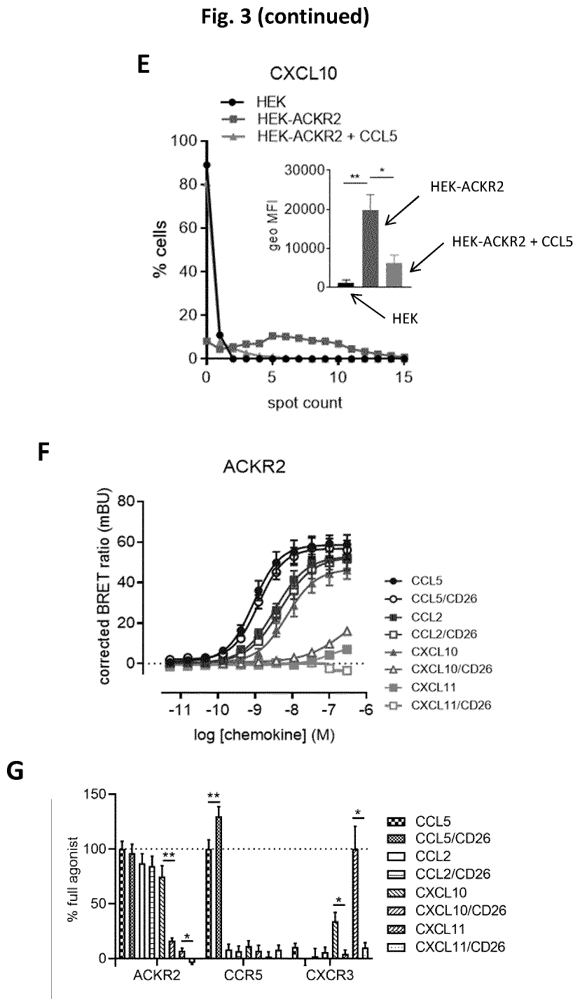

The ability of ACKR2 to scavenge CXCL10 was further evaluated by quantifying the CXCL10 in the supernatant of HEK cells stably expressing ACKR2. Imaging flow cytometry revealed a specific and efficient uptake of labelled CXCL10 by ACKR2-expressing cells. A notably higher number of distinguishable intracellular vesicle-like structures and mean fluorescent intensity were observed compared to HEK293T cells or HEK-ACKR2 cells pre-treated with CCL5 (FIG. 3F).

Importantly, following 8-hour incubation with ACKR2 positive cells, the ACKR2-driven intracellular accumulation of CXCL10 was also associated with a reduction by 70% of its availability in the extracellular space as demonstrated by ELISA quantification. This ACKR2-driven clearance of CXCL10 was comparable with its scavenging capacity towards CCL5 (FIG. 3A-D), in contrast to CXCL11, where no depletion could be observed (negative control).

Similar to many other CC and CXC chemokines, CXCL10 was shown to be subject to post-translationally modification by proteolytic enzymes. In particular, N-terminal cleavage by the dipeptidyl peptidase 4 (DPP4 or CD26) was demonstrated to turn CXCL10 from CXCR3 agonist to antagonist. Based on recent reports demonstrated that, in contrast to CXCR3, ACKR3 is still responsive to DPP4-inactivated CXCL11, the impact of the CXCL10 N-terminal processing on ACKR2 activation was evaluated and compared to CXCR3. We observed that, in contrast to CC chemokines, truncation of CXCL10 abolished its ability to induce β-arrestin-1 recruitment to ACKR2 (FIG. 3F-G) indicating that its N terminal residues are critical for ACKR2 activation. These results, in addition to partial agonist behaviour of CXCL10, point to distinct ACKR2 interactions and activation mode compared to CC chemokines. This may be attributed to notable differences in the N terminus orientation and occupation of the receptor binding pockets of CXC and CC chemokines.

ACKR2 has been so far reported to scavenge only inflammatory chemokines CC chemokines. However, present inventors showed ACKR2 is also a scavenger for CXCL10, which is an important inflammatory chemokine known to significantly contribute to the recruitment of NK and CD8+T cells into the tumor bed and to be part of the inflammatory chemokine signature require for efficient immunotherapy.

### Example 3. Targeting ACKR2 Significantly Decreases Tumor Growth and Weight of B16-F10 Melanoma

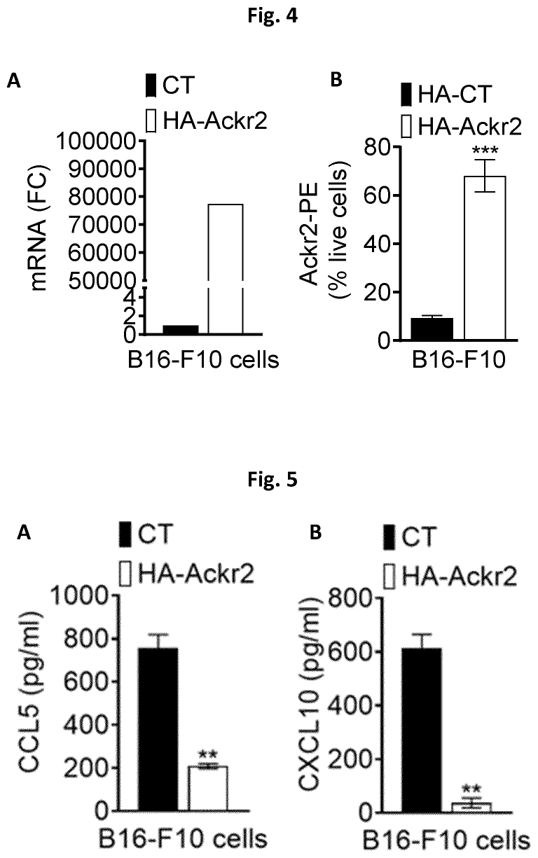

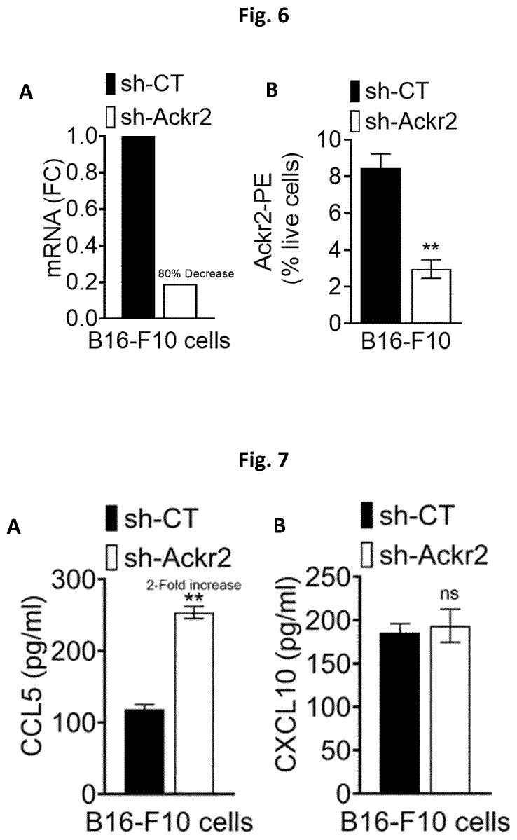

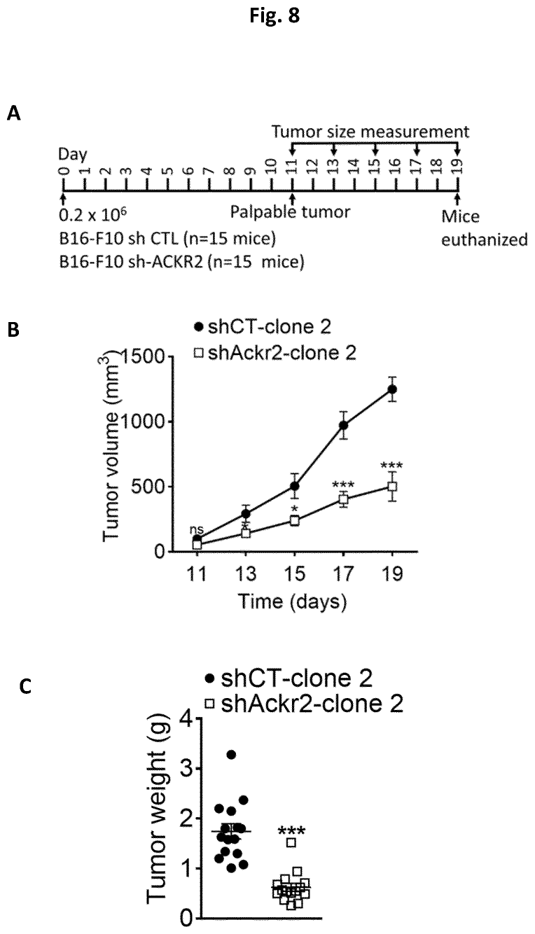

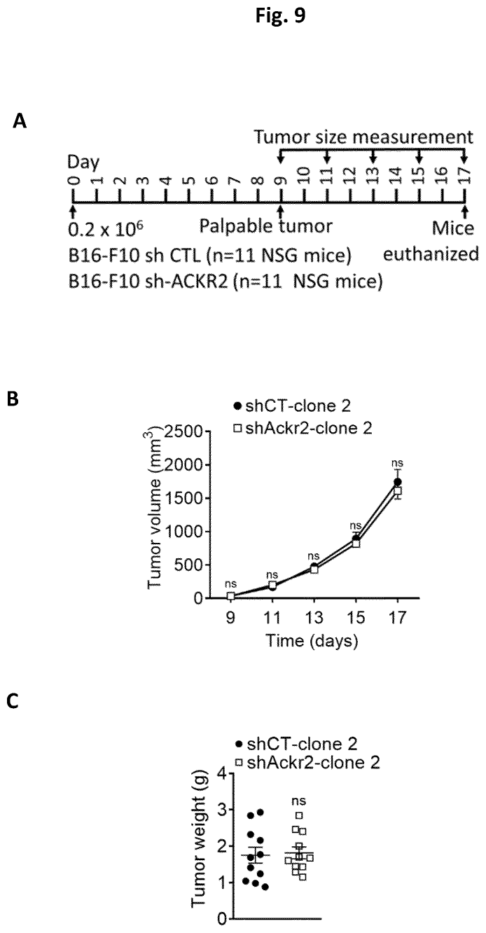

Present inventors next assessed the impact of targeting or overexpressing ACKR2 on the release of CCL5 and CXCL10 by B16-F10 melanoma cells. Briefly, present inventors showed a significant decrease in the concentration of CCL5 and CXCL10 in the supernatant of tumor cells overexpressing ACKR2. Conversely, targeting ACKR2 with shRNA led to a significant increase in the concentration of CCL5, but not CXCL10, which could be related to the preference of ACKR2 for CCL5 over CXCL10 (FIGS. 4 to 7). Furthermore, present inventors reported that targeting ACKR2 resulted in significant decrease in the B16-F10 tumor volume and weight in immunocompetent but not in immunodeficient mice (FIGS. 8 and 9).

### Example 4. Targeting ACKR2 Reprograms the Immune Landscape of B16-F10 Melanoma

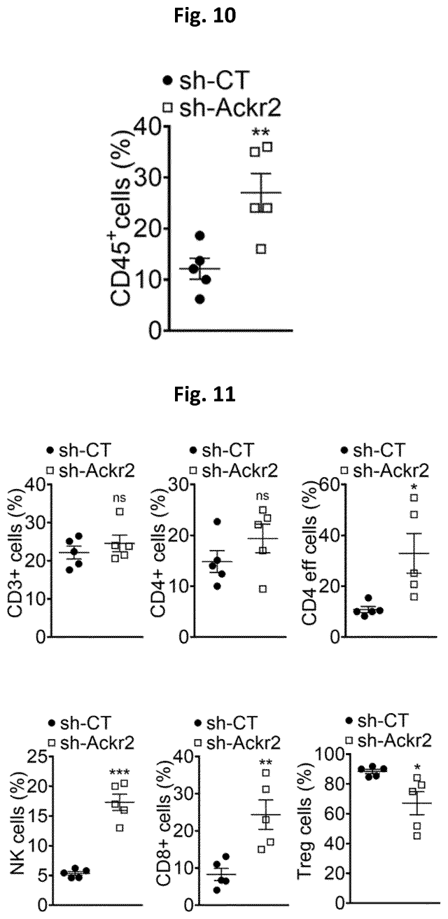

Interestingly, present inventors showed that the decrease in the volume of ACKR2 targeted B16-F10 tumors was associated with a significant increase in the infiltration of CD45+ immune cells (FIG. 10) and CD4 effector cells, NK cells and CD8 cells into the tumor microenvironment (FIG. 11). Furthermore, a significant decrease in the % of immunosuppressive Treg cells was observed in ACKR2 defective tumors and the CD8/Treg ratio was significantly increased (FIGS. 11 and 12). Present inventors also showed that the activation marker of NK and CD8 cells was also increased (FIG. 13).

Present inventors next assessed the level of CCL5, CXCL10 and IFN□ in the tumor microenvironment of control and ACKR2-targeted B16-F10 tumors. FIG. 14 shows that the level of CCL5, CXCL10 and IFNg was significantly higher in the microenvironment of ACKR2-targeted tumors compared to controls.

### Example 5. The Inhibition of Tumor Growth, Observed by Targeting Ackr2, is Dependent on the Infiltration of NK and CD8+ T Cells into the Tumor Microenvironment

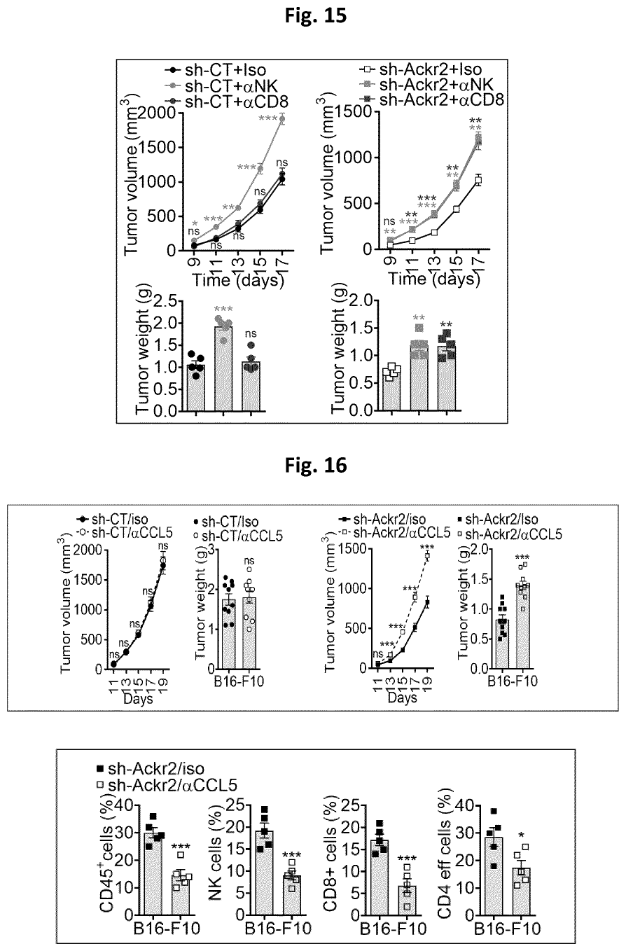

In sh-CT tumors, where limited number of NK and CD8+ T cells is present in the tumor microenvironment, depletion of CD8+ cells had no impact on tumor growth and weight. However, in sh-Ackr2 tumors, where increased number of NK and CD8+ T cells is present in the tumor microenvironment, the depletion of NK and CD8+ T cells induced a significant increase in the tumor growth and weight. These results indicate that the inhibition of B16-F10 melanoma tumor growth, observed by targeting Ackr2, depends on the recruitment of NK and CD8+ T cells into the tumor microenvironment (FIG. 15).

### Example 6. The Inhibition of Tumor Growth, Observed by Targeting Ackr2, is Dependent on the Presence of CCL5

Neutralizing the pro-inflammatory chemokine CCL5 in Ackr2 targeted (sh-Ackr2), but not in control (sh-CT) B16-F10 tumor bearing mice is sufficient to abolish the tumor inhibitory effect of Ackr2 and to prevent the infiltration of major cytotoxic immune cells in the tumor microenvironment (FIG. 16).

### Example 7. Targeting ACKR2 In Vivo Improves the Therapeutic Benefit of Anti-PD1 in Melanoma

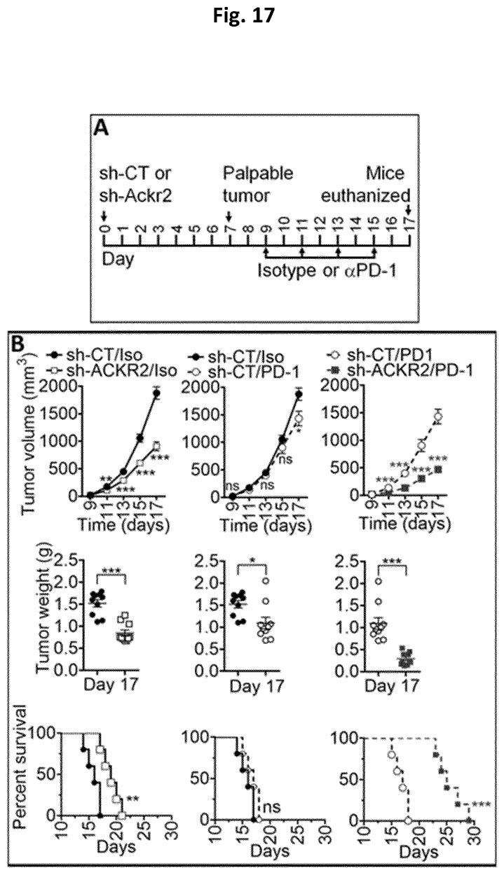

0.2×10⁶ B16-F10 cells were subcutaneously injected into the right flank of syngeneic host C57BL/6 mice at day 0. After the development of palpable tumors (typically at day 9), mice were intraperitoneally (i.p.) injected with 3 doses of 100 μg anti-PD1 or control isotype (FIG. 17a). The impact of targeting ACKR2 on the therapeutic benefit of anti-PD-1 was evaluated. FIG. 17b shows that: i) In sh-CT B16-F10 tumors, targeting Ackr2 significantly inhibits tumor growth (i.e. tumor volume) and weight and prolonged mice survival (FIG. 17b left panels); ii) In sh-CT B16-F10 tumors, anti-PD-1 treatment alone has no impact in tumor growth (i.e. tumor volume), tumor weight and mice survival (middle panels); iii) interestigly, the therapeutic benefit of anti-PD-1 treatment, which was not effective in sh-CT tumors, is significantly improved in sh-Ackr2 tumors (improvement of tumor growth inhibition, tumor weight inhibition and mice survival). These results clearly highlight that in PD-1 non-responsive tumors, targeting ACKR2 improves the therapeutic benefit of PD-1 immune checkpoint blockers, most likely by increasing the release of CCL5 and CXCL10 chemokines in the tumor microenvironment responsible for the recruitment of NK and CD8 cells.

### Example 8. Targeting ACKR2 In Vitro Using an ACKR2 Antibody Displaces CCL5 from ACKR2

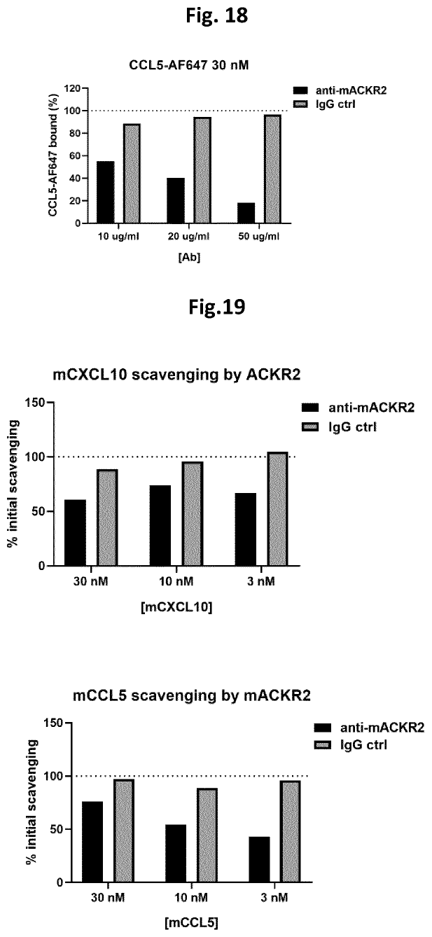

The ability of the commercially available polyclonal antibody against the murine ACKR2 (ab1656, Abcam,) to displace CCL5 from the receptor was evaluated by binding competition studies in HEK293T cells stably overexpressing ACKR2 using Alexa Fluor 647-labelled CCL5 (Almac). The results show that the antibody was able to displace CCL5-AF647 (30 nM) from ACKR2 in concentration-dependent manner with over 80% displacement achieved with 50 μg/ml of the antibody (FIG. 18).

### Example 9. Targeting ACKR2 In Vitro Using an ACKR2 Antibody Inhibits its CXCL10- and CCL5-Scavenging Function

The ability of the commercially available polyclonal antibody against the murine ACKR2 (ab1656, Abcam) to block the scavenging activity of ACKR2 was evaluated by ELISA quantification of CXCL10 and CCL5 in the supernatant of HEK293T cells stably overexpressing ACKR2. FIG. 19 shows that the antibody (20 μg/ml) was able to inhibit over 40% and 50% of CXCL10 and CCL5 scavenging, respectively.

### Example 10. Blocking ACKR2 In Vivo Using an ACKR2 Antibody Inhibits Tumor Growth and Weight and Prolongs Mice Survival

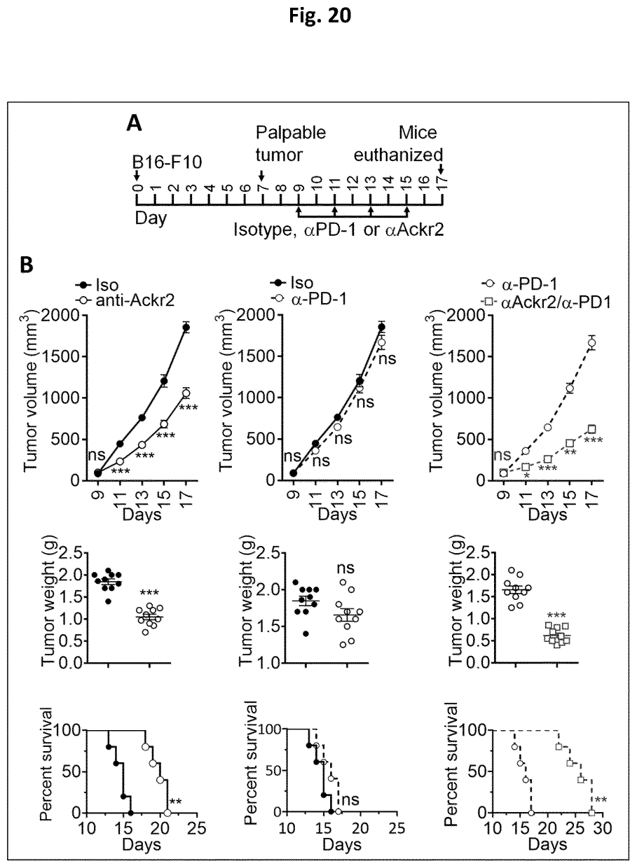

The InVivoMab anti-mouse CD8alpha (BE0061), InVivoMab anti-mouse NK1.1 (BE0036), InVivoMab anti-mouse PD-1 (CD279) (BE0273), InVivoMab rat IgG2a isotype control (BE0089) and InVivoMab rat IgG2b isotype control (BE0090) were purchased from BioXCell, diluted in InVivoPure pH 7.0 Dilution Buffer (IP0070) and administered according to the treatment strategy as indicated in FIG. 20a. Rabbit Anti-Murine RANTES (CCL5) (500-P118) was purchased from Peprotech. Goat IgG (ab37373) and polyclonal Goat anti-mACKR2 (ab1656) were purchased from Abcam.

Results showed that: i) In B16-F10 tumors, treatment with anti-Ackr2 blocking antibody significantly inhibits tumor growth and weight and prolonged mice survival (FIG. 20b; left panels); ii) In B16-F10 tumors, anti-PD-1 treatment has no impact on tumor growth, tumor weight and mice survival (FIG. 20b, middle panels); iii) The therapeutic benefit of anti-PD-1, is significantly improved in combination with the anti-Ackr2 blocking antibody, which is demonstrated by an improvement of tumor growth inhibition, tumor weight inhibition and prolonged mice survival (FIG. 20b, right panels).

1-15. (canceled)

16. A method for the treatment of a proliferative disease or disorder in a subject, said method comprising administering to said subject a specific ACKR2 modulator.

17. The method according to claim 16, wherein said treatment comprises the administration of said specific ACKR2 modulator in combination with anticancer immunotherapy.

18. The method according to claim 17, wherein said treatment comprises the administration of said specific ACKR2 modulator in combination with one or more immune checkpoint modulators.

19. The method according to claim 16, wherein said ACKR2 modulator specifically binds to ACKR2.

20. The method according to claim 16, wherein said ACKR2 modulator decreases the quantity and/or expression level of ACKR2.

21. The method according to claim 16, wherein said ACKR2 modulator is selected from a chemical substance, an antibody, an antibody fragment, an antibody-like protein scaffold, a protein or polypeptide, a peptide, a peptidomimetic, an aptamer, a photoaptamer, a spiegelmer, and a nucleic acid.

22. The method according to claim 21, wherein said ACKR2 modulator is selected from an anti-ACKR2 antibody, an ACKR2-directed gene-editing system, an RNAi agent directed against ACKR2, a fragment or derivative of CCL2, a fragment or derivative of CCL3, a fragment or derivative of CCL4, a fragment or derivative of CCL5, a fragment or derivative of CCL7, a fragment or derivative of CCL8, a fragment or derivative of CCL11, a fragment or derivative of CCL13, a fragment or derivative of CCL14, a fragment or derivative of CCL17, a fragment or derivative of CCL22, and a fragment or derivative of CXCL10.

23. The method according to claim 16, wherein said proliferative disease or disorder is a proliferative disease or disorder selected from skin cancer, colon cancer, rectal cancer, colorectal cancer, bladder cancer, neuroblastoma, squamous cell cancer, lung cancer, cancer of the peritoneum, hepatocellular cancer, gastric or stomach cancer, pancreatic cancer, glioblastoma, cervical cancer, ovarian cancer, liver cancer, hepatoma, breast cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, head cancer, and neck cancer.

24. The method according to claim 16, wherein said subject is diagnosed with or assumed to have a proliferative disease or disorder which is non-responsive to anticancer immunotherapy.

25. The method according to claim 16, wherein said subject is diagnosed with or assumed to have a proliferative disease or disorder which is non-responsive to immune checkpoint blockade therapy.

26. The method according to claim 17, wherein the one or more immune checkpoint modulators are selected from a Programmed Death-ligand 1 (PDL-1) inhibitor, a Programmed Death 1 (PD-1) inhibitor, a Cytotoxic T-Lymphocyte-Associated protein 4 (CTLA-4) inhibitor, a cluster of differentiation 3 (CD3) inhibitor, a NKG2A inhibitor, a immunoglobulin-like receptors (KIR) inhibitor, a cluster of differentiation 47 (CD47) inhibitor, a CD24 inhibitor, a CD73 inhibitor, a tumor necrosis factor (TNF) receptor 2 (TNFR2) inhibitor, and a signal-regulatory protein alpha (SIRPα) inhibitor.

27. The method according to claim 26, wherein the PD-1 inhibitor is selected from nivolumab, pembrolizumab, pidilizumab, cemiplimab, tislelizumab, sintilimab, and MEDI068.

28. A pharmaceutical composition comprising a specific ACKR2 modulator and one or more immune checkpoint modulators and optionally a pharmaceutically acceptable carrier.

29. The pharmaceutical composition according to claim 28, wherein the one or more immune checkpoint modulators.

30. The pharmaceutical composition according to claim 29, wherein the one or more immune checkpoint modulators are selected from a Programmed Death-ligand 1 (PDL-1) inhibitor, a Programmed Death 1 (PD-1) inhibitor, a Cytotoxic T-Lymphocyte-Associated protein 4 (CTLA-4) inhibitor, a cluster of differentiation 3 (CD3) inhibitor, a NKG2A inhibitor, a immunoglobulin-like receptors (KIR) inhibitor, a cluster of differentiation 47 (CD47) inhibitor, a CD24 inhibitor, a CD73 inhibitor, a tumor necrosis factor (TNF) receptor 2 (TNFR2) inhibitor, and a signal-regulatory protein alpha (SIRPα) inhibitor.

31. The pharmaceutical composition according to claim 30, wherein the PD-1 inhibitor is selected from nivolumab, pembrolizumab, pidilizumab, cemiplimab, tislelizumab, sintilimab, and MEDI068.

32. An in vitro method for identifying an agent useful as a therapeutic, said method comprising determining whether a test agent modulates the biological activity of ACKR2.

33. The in vitro method according to claim 32, wherein said method comprises a step of determining whether a test agent decreases or eliminates the biological activity of ACKR2.

34. The in vitro method according to claim 32, wherein said method comprises
contacting the test agent with a cell capable inducing β-arrestin 1 and/or β-arrestin 2 recruitment to ACKR2 in the presence of CCL2, CCL3, CCL4, CCL5, CCL7, CCL8, CCL11, CCL13, CCL14, CCL17, CCL22 and/or CXCL10 and measuring β-arrestin 1 and/or β-arrestin 2 recruitment to ACKR2, and
determining that the agent is useful as a therapeutic when the agent reduces or eliminates β-arrestin1 and/or β-arrestin 2 recruitment to ACKR2 in the presence of CCL2, CCL3, CCL4, CCL5, CCL7, CCL8, CCL11, CCL13, CCL14, CCL17, CCL22 and/or CXCL10.

35. The in vitro method according to claim 32, wherein said method further comprises determining whether the test agent specifically binds to ACKR2.


SEQUENCE LISTING (3 sequences)

SEQ ID NO: 1 - protein, 50 aa, Homo sapiens
MAATASPQPLATEDADSENSSFYYYDYLDEVAFMLCRKDAVVSFGKVFLP

SEQ ID NO: 2 - protein, 34 aa, Homo sapiens
QTHENPKGVWNCHADFGGHGTIWKLFLRFQQNLL

SEQ ID NO: 3 - protein, 22 aa, Homo sapiens
LHTLLDLQVFGNCEVSQHLDYA